(19) United States
(12) Patent Application Publication
(10) Pub. No.: US 2022/0325266 A1
(43) Pub. Date: Oct. 13, 2022

(54) SYNTHETIC GENES FOR THE TREATMENT OF PROPIONIC ACIDEMIA CAUSED BY MUTATIONS IN PROPIONYL-COA CARBOXYLASE BETA

(71) Applicant: The United States of America,as represented by the Secretary,Department of Health and Human Services, Bethesda, MD (US)

(72) Inventors: Charles P. Venditti, Potomac, MD (US); Randy J. Chandler, Chevy Chase, MD (US)

(73) Assignee: The United States of America,as represented by the Secretary,Department of Health and Human Services, Bethesda, MD (US)

(21) Appl. No.: 17/766,067

(22) PCT Filed: Oct. 15, 2020
(86) PCT No.: PCT/US2020/055836

§ 371 Date: Apr. 1, 2022

Related U.S. Application Data
(60) Provisional application No. 62/915,347, filed on Oct. 15, 2019.

(51) Int. Cl. C12N 9/00; C12N 15/86

(52) U.S. Cl. CPC C12N 9/93; C12Y 604/01003; C12N 15/86; C12N 2750/14143; C12N 2840/105; C12N 2830/50; C12N 2800/22; A61K 48/00

(57) ABSTRACT

Synthetic polynucleotides encoding human propionyl-CoA carboxylase beta (synPCCB) and exhibiting augmented expression in cell culture and/or in a subject are described herein. An adeno-associated viral (AAV) gene therapy vector encoding synPCCB successfully rescued the neonatal lethal phenotype displayed by propionyl-CoA carboxylase beta (Pccb^{-/-}) deficient mice, lowered circulating methylcitrate levels in the treated animals, and resulted in prolonged hepatic expression of the product of the synPCCB transgene in vivo.

### RELATED APPLICATIONS

This application claims the benefit of U.S. Provisional Application Ser. No. 62/915,347, filed Oct. 15, 2019 which is incorporated by reference in its entirety.

### STATEMENT OF GOVERNMENT INTEREST

The instant application was made with government support; the government has certain rights in this invention.

### SEQUENCE LISTING DATA

The Sequence Listing text document filed herewith, created Oct. 15, 2020, size 129,036 bytes, and named “750505_ST25.txt,” is incorporated herein by reference in its entirety.

### FIELD OF THE INVENTION

The subject invention relates to engineering of the human propionyl-CoA carboxylase beta gene (PCCB) so as to enhance its expression in eukaryotic cells. Compared to the wild-type human PCCB gene, the subject synthetic gene sequences (synPCCB) are codon-optimized to enhance expression upon administration.

### BACKGROUND

Propionic acidemia (PA) is an autosomal recessive metabolic disorder caused by mutations in either of PCCA or PCCB genes. The products of these genes form the alpha and beta subunits of the enzyme propionyl-CoA carboxylase (PCC), a critically important mitochondrial enzyme involved in the catabolism of branched chain amino acids. Specifically, propionyl-CoA carboxylase catalyzes the carboxylation of propionyl-CoA to D-methylmalonyl-CoA.

The results from an ongoing PA natural history study have revealed that in a large and diverse cohort of patients, approximately 50% have PA caused by PCCB mutations. Many PA patients present within the first few days to weeks of life with symptoms, and lethality can ensue if clinical recognition and treatment is delayed. Laboratory investigations show characteristic elevations of propionylcarnitine, 3-hydroxypropionate, and 2-methylcitrate (MC). Milder patients can escape from early presentations but remain at risk for metabolic decompensation and late complications, especially cardiomyopathy. All individuals with PA can experience high mortality and disease related morbidity despite nutritional therapy. The failure of conventional medical and dietary management to treat PA has led to the use of elective liver transplantation as an alternative approach to stabilize metabolism and mitigate the risk of lethal metabolic decompensations.

### SUMMARY

The only treatments for PA currently available are dietary restrictions and elective liver transplantation. Patients still become metabolically unstable while on diet restriction and experience disease progression, despite medical therapy. These episodes result in numerous hospitalizations and can be fatal. A synthetic human propionyl-CoA carboxylase beta (synPCCB) transgene can be used as a drug, via viral- or non-viral mediated gene delivery, to restore PCC function in some PA patients, prevent metabolic instability, and ameliorate disease progression. Because this enzyme may also be important in other disorders of branched chain amino acid oxidation, gene delivery of synthetic PCCB gene could be used to treat conditions other than PA.

Additionally, the synPCCB transgene can be used for the in vitro production of PA for use in enzyme replacement therapy for PA. Enzyme replacement therapy is accomplished by administration of the synthetic PCC protein orally, sub-cutaneously, intra-muscularly, intravenously, or by other therapeutic delivery routes.

Thus, in one aspect, the invention is directed to a synthetic propionyl-CoA carboxylase beta gene (synPCCB) selected from the group consisting of:

a) a polynucleotide comprising the nucleic acid sequence of any one of SEQ ID NOs:2-6;
b) a polynucleotide having the nucleic acid sequence of any one of SEQ ID NOs:2-6;
c) a polynucleotide having a nucleic acid sequence with at least about 80% identity to the nucleic acid sequence of any one of SEQ ID NOs:2-6;
d) a polynucleotide encoding a polypeptide having the amino acid sequence of SEQ ID NO:7 or an amino acid sequence substantially identical to the amino acid sequence of SEQ ID NO:7, wherein the polynucleotide does not have the nucleic acid sequence of SEQ ID NO:1; and
e) a polynucleotide encoding an active fragment of the propionyl-CoA carboxylase (PCC) protein, wherein the polynucleotide in its entirety does not share 100% identity with a portion of the nucleic acid sequence of SEQ ID NO:1.

In one embodiment, the fragment includes only amino acid residues encoded by synPCCB, and which represents the active, processed form of PCCB.

By active, it can be meant, for example, the enzyme's ability to catalyze the carboxylation of propionyl-CoA to D-methylmalonyl CoA. The activity can be assayed using methods well-known in the art (as described in the context of protein function, below).

In one embodiment of a synthetic polynucleotide according to the invention, the nucleic acid sequence encodes a polypeptide having the amino acid sequence of SEQ ID NO:7 or an amino acid sequence with at least about 90% identity to the amino acid sequence of SEQ ID NO:7.

In another embodiment, the synthetic polynucleotide exhibits augmented expression relative to the expression of naturally occurring human propionyl-CoA carboxylase beta polynucleotide sequence (SEQ ID NO:1) in a subject. In yet another embodiment, the synthetic polynucleotide having augmented expression comprises a nucleic acid sequence comprising codons that have been optimized relative to the naturally occurring human propionyl-CoA carboxylase beta polynucleotide sequence (SEQ ID NO:1). In still another embodiment of a synthetic polynucleotide according to the invention, the nucleic acid sequence has at least about 80% of less commonly used codons replaced with more commonly used codons.

In one embodiment of a synthetic polynucleotide according to the invention, the polynucleotide is a polynucleotide having a nucleic acid sequence with at least about 85% identity to the nucleic acid sequence of any one of SEQ ID NOs: 2-6. In another embodiment, the polynucleotide is a polynucleotide having a nucleic acid sequence with at least about 90% identity to the nucleic acid sequence of any one of SEQ ID NOs: 2-6. In still another embodiment, the polynucleotide is a polynucleotide having a nucleic acid sequence with at least about 95% identity to the nucleic acid sequence of any one of SEQ ID NOs: 2-6.

In one embodiment of a synthetic polynucleotide according to the invention, the nucleic acid sequence is a DNA sequence. In another embodiment, the nucleic acid sequence is a RNA sequence, such as mRNA, or peptide modified nucleic acid sequence. In another embodiment, the synthetic polynucleotide according to the invention encodes an active PCCB subunit.

In another aspect, the invention is directed to an expression vector comprising the herein-described synthetic polynucleotide. In another embodiment of a vector according to the invention, the synthetic polynucleotide is operably linked to an expression control sequence. In still another embodiment, the synthetic polynucleotide is codon-optimized.

In a further aspect, the invention is directed to a method of treating a disease or condition mediated by propionyl-CoA carboxylase or low levels of propionyl-CoA carboxylase activity, the method comprising administering to a subject the herein-described synthetic polynucleotide.

In still a further aspect, the invention is directed to a method of treating a disease or condition mediated by propionyl-CoA carboxylase, the method comprising administering to a subject a propionyl-CoA carboxylase produced using the synthetic polynucleotide described herein. In another embodiment of a method of treatment according to the invention, the disease or condition is propionic acidemia (PA).

In one aspect, the invention is directed to a composition comprising a synthetic polynucleotide and a pharmaceutically acceptable carrier.

In another aspect, the invention is directed to a composition comprising a synthetic polynucleotide, a pharmaceutically acceptable carrier and a polynucleotide encoding the propionyl-CoA carboxylase beta subunit.

In another aspect, the invention is directed to a transgenic animal whose genome comprises a polynucleotide sequence encoding propionyl-CoA carboxylase beta or a functional fragment thereof. In still another aspect, the invention is directed to a method for producing such a transgenic animal, comprising: providing an exogenous expression vector comprising a polynucleotide comprising a promoter operably linked to a polynucleotide encoding propionyl-CoA carboxylase beta or a functional fragment thereof; introducing the vector into a fertilized oocyte; and transplanting the oocyte into a female animal.

In one aspect, the invention is directed to a transgenic animal whose genome comprises the synthetic polynucleotide described herein. In another aspect, the invention is directed to a method for producing such a transgenic animal, comprising: providing an exogenous expression vector comprising a polynucleotide comprising a promoter operably linked to the synthetic polynucleotide described herein; introducing the vector into a fertilized oocyte; and transplanting the oocyte into a female animal.

Methods for producing transgenic animals are known in the art and include, without limitation, transforming embryonic stem cells in tissue culture, injecting the transgene into the pronucleus of a fertilized animal egg (DNA microinjection), genetic/genome engineering, viral delivery (for example, retrovirus-mediated gene transfer).

Transgenic animals according to the invention include, without limitation, rodent (mouse, rat, squirrel, guinea pig, hamster, beaver, porcupine), frog, ferret, rabbit, chicken, pig, sheep, goat, cow primate, and the like.

In another aspect, the invention is directed to the preclinical amelioration or rescue from the disease state, for example, propionic acidemia, that the afflicted subject exhibits. This may include symptoms, such as lethargy, lethality, metabolic acidosis, and biochemical perturbations, such as increased levels of methylcitrate in blood, urine, and body fluids.

In still another aspect, the invention is directed to a method for producing a genetically engineered animal as a source of recombinant synPCCB. In another aspect, genome editing, or genome editing with engineered nucleases (GEEN) may be performed with the synPCCB nucleotides of the present invention allowing synPCCB DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the nucleotides of the present invention including synPCCB, in combination with a CASP/CRISPR, ZFN, or TALEN can be used to engineer correction at the locus in a patient's cell either in vivo or ex vivo, then, in one embodiment, use that corrected cell, such as a fibroblast or lymphoblast, to create an iPS or other stem cell for use in cellular therapy.

In yet another embodiment, the invention is directed at the production of viral vectors designed to express the polynucleotides as described above. Examples of vectors include, but are not limited to, adenoassociated viral vectors (AAV), adenoviral vectors, retroviral vectors, lentiviral vectors, and herpes viral vectors. Examples of AAV serotypes include, but are not limited to AAV1, 2, 3, 3B, 4, 5, 6, 7, 8, 9, 44.9, rh8, rh10, ANC80L65, LK03, NP59, KP1, and others well known to practitioners of the art.

Hybrid vectors that use a combination of AAV vectors and vectors, including non viral vectors such as lipid nanoparticles (LNPs), designed to deliver and express Piggy Bac (PB) transposons or other DNA transposons that use a cut and paste mechanism to accomplish insertion of the target sequence is yet another embodiment of the use of the polynucleotides of described herein. Hybrid vectors can include a AAV designed to express a viral transgene encoding the PCCB polynucleotide(s), either alone or in combination with a polynucleotide encoding the propionyl-CoA carboxylase alpha, and flanked by Piggy Bac (PB) transposon specific terminal repeats, when administered with a source of Piggy Bac (PB) transpose mRNA, will enable transposition of the viral transgene into the genome, with permanent correction of transduced cells resulting. Such cells include hepatocytes, the main cell of the liver, which is targeted by many AAV serotypes, AAV1, 2, 3, 3B, 4, 5, 6, 7, 8, 9, 44.9, rh8, rh10, ANC80L65, LK03, NP59, KP1, and others well known to practitioners of the art.

In yet another embodiment, the invention is directed at the production of viral vectors designed to express the PCCB polynucleotide(s) and a polynucleotide encoding the propionyl-CoA carboxylase alpha. Examples of vectors include, but are not limited to, adenoassociated viral vectors (AAV), adenoviral vectors, retroviral vectors, lentiviral vectors, and herpes viral vectors. Examples of AAV serotypes include, but are not limited to AAV1, 2, 3, 3B, 4, 5, 6, 7, 8, 9, 44.9, rh8, rh10, ANC80L65, LK03, NP59, KP1, and others well known to practitioners of the art.

In yet another embodiment, the invention is directed at the production of lipid nanoparticles designed to express the PCCB polynucleotide(s) and a polynucleotide encoding the propionyl-CoA carboxylase alpha.

In yet another embodiment, the invention is directed at the production of DNA plasmids designed to express the PCCB polynucleotide(s) and a polynucleotide encoding the propionyl-CoA carboxylase alpha. Such plasmids may be devoid of bacterial DNA and include forms of closed end DNA generated by AAV replication in a baculoviral system.

The following numbered paragraphs [0032]-[0087] contain statement of broad combination of the inventive technical features disclosed herein:

1. A synthetic propionyl-CoA carboxylase subunit beta (PCCB) polynucleotide (synPCCB) selected from the group consisting of:
a) a polynucleotide comprising the nucleic acid sequence of any one of SEQ ID NOs: 2-6; and
b) a polynucleotide having a nucleic acid sequence with at least about 80% identity to the nucleic acid sequence of any one of SEQ ID NOs: 2-6 and encoding a polypeptide according to SEQ ID NO:7, and having equivalent or increased expression in a host as compared to either expression of any one of SEQ ID NOs: 2-6 or SEQ ID NO:1 expression, wherein the polynucleotide does not have the nucleic acid sequence of SEQ ID NO:1.

2. The synthetic polynucleotide of aspect 1, wherein the polynucleotide has at least about 90% identity to the nucleic acid sequence of any one of SEQ ID NOs: 2-6.

3. The synthetic polynucleotide of aspect 1, wherein the polynucleotide has at least about 95% identity to the nucleic acid sequence of any one of SEQ ID NOs: 2-6.

4. The synthetic polynucleotide of aspect 1, wherein the sequence selected from the group consisting of SEQ ID NOs: 2-6 exhibits increased expression in an appropriate host relative to the expression of SEQ ID NO:1 in an appropriate host.

5. The synthetic polynucleotide of aspect 3, wherein the synthetic polynucleotide having increased expression comprises a nucleic acid sequence comprising codons that have been optimized relative to the naturally occurring human propionyl-CoA carboxylase subunit beta polynucleotide sequence (SEQ ID NO:1).

6. The synthetic polynucleotide of aspect 4, wherein the nucleic acid sequence has at least about 70% of less commonly used codons replaced with more commonly used codons.

7. A recombinant expression vector comprising the synthetic polynucleotide of any one of aspects 1-6.

8. The recombinant vector of aspect 7, wherein the vector is a recombinant adeno-associated virus (rAAV), said rAAV comprising an AAV capsid, and a vector genome packaged therein, said vector genome comprising:

(a) a 5′-inverted terminal repeat sequence (5′-ITR) sequence;
(b) a promoter sequence;
(c) a partial fragment or complete coding sequence for PCCB; and
(d) a 3′-inverted terminal repeat sequence (3′-ITR) sequence.

9. The rAAV according to aspect 8, wherein the vector is comprised of the structure in FIG. 1.

10. The rAAV according to aspect 7 or 8, wherein the AAV capsid is from an AAV of serotype AAV1, 2, 3, 3B, 4, 5, 6, 7, 8, 9, 44.9, rh8, rh10, ANC80L65, LK03, NP59, KP1, and mutants thereof.

11. The rAAV according to aspect 8, wherein the AAV capsid is from an AAV of serotype 8.

12. The rAAV according to aspect 8, wherein the AAV capsid is from an AAV of serotype 9.

13. The rAAV according to aspect 7 or 8, further comprising terminal repeat sequences recognized by piggyBac transposase.

14. The rAAV according to any one of aspects 8-12, wherein the promoter is selected from the group consisting of chicken-beta actin promoter (SEQ ID NO: 8), the elongation factor 1 alpha long promoter (EF1AL) (SEQ ID NO:9), the elongation factor 1 alpha short promoter with a 3′ hepatitis B post translation response element (HPRE) (SEQ ID NO:10), and human alpha 1 antitrypsin promoter with a 3′ woodchuck post translation response element (WPRE) (SEQ ID NO:11).

15. The rAAV according to any one of aspects 8-12, wherein the promoter is selected from the group consisting of liver specific enhancer and promoter, such as the long (SEQ ID NO:13), or short variants (SEQ ID NO:12) of the apolipoprotein E enhancer, and further comprising operably linked to the long (SEQ ID NO:15) or short variants of the human alpha 1 antitrypsin promoter (SEQ ID NO:14), and optionally at least one intron selected from the group consisting of a chimeric intron (SEQ ID NO:16), modified B-globin intron (SEQ ID NO: 17), and a synthetic intron (SEQ ID NO:18).

16. The rAAV according to any one of aspects 8-11, wherein the promoter is selected from the group consisting of a liver specific enhancer and promoters of a long (SEQ ID NO:13), or short variant (SEQ ID NO:12) of the apolipoprotein E enhancer, the enhanced human alpha 1 antitrypsin promoter (SEQ ID:35), and the enhanced TBG promoter (SEQ ID:34), further comprising operably linked to the long (SEQ ID NO:15) or short variants of the human alpha 1 antitrypsin promoter (SEQ ID NO:14) and followed by either a chimeric intron (SEQ ID NO:16), modified B-globin intron (SEQ ID NO: 17), or a synthetic intron (SEQ ID NO:18).

17. The rAAV according to aspect 16, wherein the apolipoprotein E enhancer, and the human alpha 1 antitrypsin promoter are operably linked to form a short (SEQ ID NO: 19) or long liver specific enhancer-promoter units (SEQ ID NO: 20) and placed 5′ to an intron selected from SEQ ID NO:16-18.

18. The rAAV according to aspect 17, wherein the intron is the modified-globin intron (SEQ ID NO: 17).

19. The rAAV according to aspect 17, wherein the intron comprises a polynucleotide having the sequence of SEQ ID NO:21.

20. The rAAV according to aspect 16, wherein the liver specific enhancer is derived from sequences upstream of the alpha-1-microglobulin/bikunin precursor (SEQ ID:22 and SEQ ID:23), and operably linked to the human thyroxine-binding globulin promoter (TBG) (SEQ ID:24).

21. The rAAV according to aspect 16, wherein the liver specific enhancer and human thyroxine-binding globulin promoter is SEQ ID:25.

22. The synthetic polynucleotide of any one of aspects 1-6, wherein the synthetic PCCB gene is flanked by a 5′ untranslated region (5′UTR) that includes a strong Kozak translational initiation signal.

23. The synthetic polynucleotide of aspect 22, further comprising an internal ribosome entry site (IRES) (SEQ ID: 26) instead of, or in addition to, a UTR.

24. The synthetic polynucleotide of aspect 23, further comprising at least one translation enhancer element (TEE).

25. The synthetic polynucleotide of aspect 24, wherein the TEE is located between the promoter and the start codon.

26. The synthetic polynucleotide of aspect 24, wherein the 5′UTR comprises a TEE.

27. The synthetic polynucleotide of aspect 22, wherein the UTR comprises sequences selected from the group consisting of human albumin (SEQ ID: 27), SERPINA 1 (SEQ ID: 28), and SERPINA 3 (SEQ ID: 29).

28. The synthetic polynucleotide of any one of aspects 1-6, wherein the polynucleotide further comprises the wood chuck post-translational response element (SEQ ID: 30) or the sequence comprises the hepatitis post-translational response element (SEQ ID:31).

29. The synthetic polynucleotide of any one of aspects 1-6, further comprising a polyadenylation signal.

30. The synthetic polynucleotide of aspect 29, wherein the polyadenylation signal is a rabbit beta globin gene or the bovine growth hormone gene.

31. The rAAV according to any one of aspects 8-11, further comprises terminal repeat sequences (SEQ ID: 32-33) from the piggyBac transposon, located after the 5′AAV ITR and before the 3′ AAV ITR.

32. The synthetic polynucleotide of aspects 1-6, further comprising a donor cassette that targets the stop codon of human albumin, which yields, after homologous recombination synPCCB1 fused via a P2 peptide to the carboxy terminus of albumin.

33. The synthetic polynucleotide of any one of aspects 1-6, further comprising an integrating AAV vector that uses homologous recombination to insert synPCCB1 into human albumin (SEQ ID NO: 36).

34. The synthetic polynucleotide of aspect 32, further comprising an integrating AAV vector that uses homologous recombination to insert synPCCB1 into the 5′ end of human albumin (SEQ ID NO: 37).

35. The synthetic polynucleotide of any one of aspects 1-6, wherein the synthetic PCCB gene is configured to integrate into the genome after delivery using a lentiviral vector.

36. The synthetic polynucleotide of aspect 35, wherein lentiviral vector further comprises a human alpha 1 antitrypsin enhancer having the sequence SEQ ID:38.

37. The synthetic polynucleotide of aspect 35, wherein the lentiviral vector further comprises the elongation factor 1 long promoter having the sequence SEQ ID:39.

38. The vector of any one of aspect 8-13, wherein the promotor is a tissue specific promoter.

39. The vector of aspect 38, wherein the tissue specific promoter promotor is selected from the group consisting of Apo A-I, ApoE, hAAT, transthyretin, liver-enriched activator, albumin, TBG, PEPCK, and RNAPII promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, prolactin, and myelin basic protein promoters (neuronal), and ankyrin, α-spectrin, globin, HLA-DRα, CD4, glucose 6-phosphatase, and dectin-2 promoters (erythroid).

40. The expression vector of aspect 7 or 8, wherein the expression vector is AAV2/9-CBA-synPCCB1.

41. The expression vector of aspect 7 or 8, wherein the expression vector is AAV2/9-EF1L-synPCCB1.

42. The expression vector of aspect 7 or 8, wherein the expression vector is AAV2/9-EF1S-HPRE synPCCB1.

43. The expression vector of aspect 7 or 8, wherein the expression vector is AAV2/9-hAAT-WPRE synPCCB1 or AAV2/9-HCR-hAAT-synPCCB1 RBG.

44. A composition comprising the synthetic polynucleotide of any one of aspects 1-6 and a pharmaceutically acceptable carrier.

45. The composition of aspect 44, further comprising the expression vector of any one of aspects 7-13 and a pharmaceutically acceptable carrier.

46. The composition of aspect 13, further comprises a hybrid AAV-piggyBac transposon system.

47. A method of treating a disease or condition mediated by propionyl-CoA carboxylase, comprising administering to a subject in need thereof a therapeutic amount of the synthetic polynucleotide of any one of aspects 1-6.

48. A method of treating a disease or condition mediated by propionyl-CoA carboxylase, comprising administering to a subject a propionyl-CoA carboxylase produced using the synthetic polynucleotide of any one of aspects 1-6.

49. The method of any one of aspects 47-48, wherein the disease or condition is propionic acidemia (PA).

50. The method of treating a disease or condition mediated by propionyl-CoA carboxylase of any one of aspects 48-49, comprising administering to a cell of a subject in need thereof the polynucleotide of aspect 1, wherein the polynucleotide is inserted into the cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system) and meganuclease re-engineered homing endonucleases on a cell from the subject; and administering the cell to the subject.

51. A method of treating a disease or condition mediated by propionyl-CoA carboxylase, comprising administering to a subject a propionyl-CoA carboxylase produced using the rAAV of any one of aspects 7-21.

52. The method of aspect 48 wherein the composition is administered through the route consisting of subcutaneously, intramuscularly, intradermally, intraperitoneally, and intravenously.

53. The method of aspect 52 wherein the composition is administered through the route consisting of subcutaneously, intramuscularly, intradermally, intraperitoneally, and intravenously.

54. The method of any one of aspects 25-27, wherein the rAAV is administered at a dose of about 1×1011 to about 1×1014 genome copies (GC)/kg.

55. The method according to any of aspects 25-27, wherein administering the rAAV comprises administration of a single dose of rAAV.

56. The method according to any of aspects 25-27, wherein administering the rAAV comprises administration of multiple doses of rAAV.

### BRIEF DESCRIPTION OF THE DRAWINGS

FIG. 1 is a schematic of a representative AAV vector genome showing the hypothetical placement of the various vector elements. ITR—inverted terminal repeats; E-enhancer; P-promoter; I-intron; 5′ UTR/TEE—untranslated region/translational enhancer element; ATG-start codon; MMUT-human methymalonyl-coa mutase gene; TAA-stop codon; HPRE-Hepatitis B virus posttranscriptional regulatory element; POLYA-polyadenylation sequence; ITR-inverted terminal repeat.

FIG. 2 is an illustrative diagram showing transgenes that use an EF1a promoter to be packaged using wild-type adeno associated virus (AAV) serotype 8 or 9 capsids.

FIG. 3 is an illustrative diagram showing transgenes that use a hAAT promoter to be packaged using wild-type adeno associated virus (AAV) serotype 8 or 9 capsids.

FIG. 4A presents the percent sequence identity of different PCCB alleles versus wild type PCCB, and each other, showing that all the synPCCB sequences (SEQ ID NOs: 2-6) differ from the wild type PCCB gene (SEQ ID NO: 1) by >18% at the nucleotide level, and similarly, diverge from each other between 15-25%.

FIG. 4B shows the ClustalW weighted sequence distances of the synPCCB sequences (SEQ ID NOs: 2-6) and the wild type PCCB gene (SEQ ID NO: 1) using a phylogenetic analysis where distinct grouping is apparent.

FIG. 5 shows the list of codon frequencies in the human proteome.

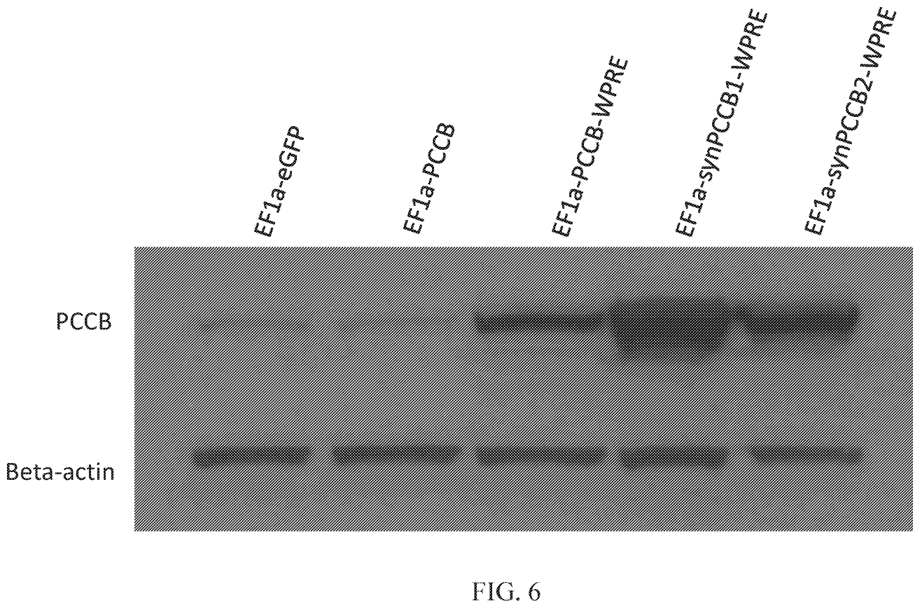

FIG. 6 presents a western blot of 293 cells transfected with equal amounts of propionyl-CoA carboxylase (PCCB) plasmid DNA from a wild-type human PCCB, and synPCCB1 and synPCCB2 expression cassettes, which used the Elongation factor 1-alpha (EF1a) promoter to drive expression. The codon optimized synPCCB1 cDNA and synPCCB2 cDNA show greater PCCB protein expression than wild-type PCCB cDNA.

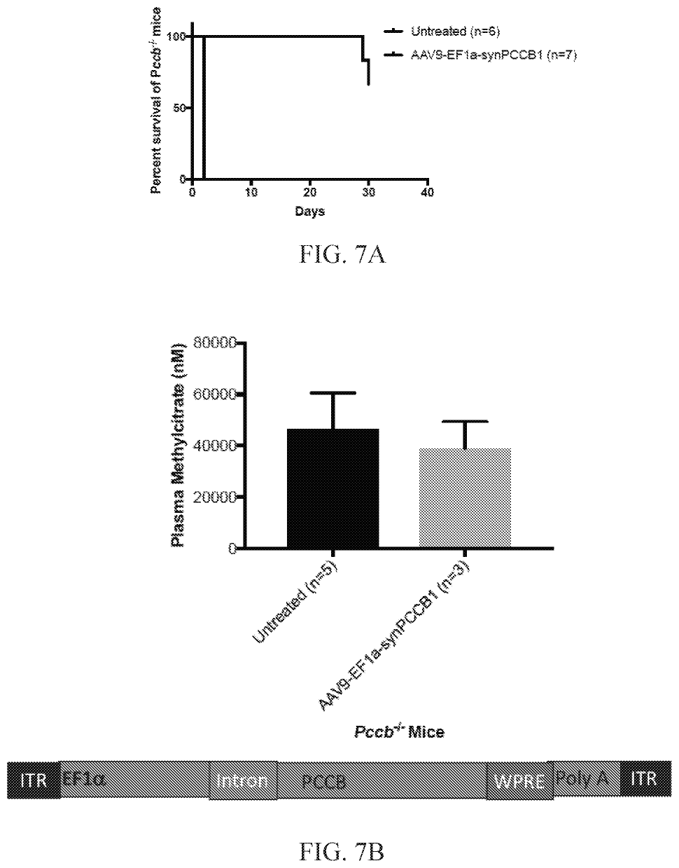

FIG. 7A presents survival data for untreated Pccb^{-/-} (n=6) mice compared to Pccb^{-/-} mice (n=7) treated with 1×10¹¹ VC of AAV-EF1a-synPCCB1 delivered by intrahepatic injection at birth. Treated Pccb^{-/-} mice display a significant increase in survival and were indistinguishable from their wild-type litter mates.

FIG. 7B presents plasma methylcitrate levels in untreated Pccb^{-/-} (n=3) mice and Pccb^{-/-} mice (n=5) treated with 1×10¹¹ VC of AAV-EF1a-synPCCB1 by intrahepatic injection at birth. A schematic of the vector map is presented in FIG. 7B. Treated Pccb^{-/-} mice have a decrease in the disease related biomarker, methylcitrate.

FIGS. 8A-8C Survival in untreated Pccb^{-/-} (n=6, called Pccb Ala499Del) mice compared to Pccb^{-/-} mice (n=5) treated with 3×10¹¹ VC of AAV-hAAT-synPCCB1 delivered by intrahepatic injection at birth. A schematic of the vector map is presented in FIG. 8A. (A) Treated Pccb^{-/-} mice display a significant increase in survival and were indistinguishable from their wild-type litter mates. (B) Plasma methylcitrate levels in untreated Pccb^{-/-} mice (n=6) treated with 3×10¹¹ VC of AAV-hAAT-synPCCB1 by intrahepatic injection at birth. Treated Pccb^{-/-} mice have a decrease in the disease related biomarker, methylcitrate. (C) Treated Pccb^{-/-} mice display a significant increase in weight, which is normalizes as the animals age.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

### Definitions

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices, and materials are now described.

As used in this application, including the appended claims, the singular forms “a,” “an,” and “the” include plural references, unless the content clearly dictates otherwise, and are used interchangeably with “at least one” and “one or more.” Thus, reference to “a polynucleotide” includes a plurality of polynucleotides or genes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

In the context of synPCCB, the terms “gene” and “transgene” are used interchangeably. A “transgene” is a gene that has been transferred from one organism to another.

The term “subject”, as used herein, refers to a domesticated animal, a farm animal, a primate, a mammal, for example, a human.

The phrase “substantially identical”, as used herein, refers to an amino acid sequence exhibiting high identity with a reference amino acid sequence (for example, at least 80%, at least 85%, at least 90%, at least 95%, at least 98%, or at least 99% identity) and retaining the biological activity of interest (the enzyme activity).

The polynucleotide sequences encoding the beta subunit of PCC, synPCCB, allow for increased expression of the synPCCB gene relative to naturally occurring human PCCB sequences. These polynucleotide sequences are designed to not alter the naturally occurring human PCC beta subunit amino acid sequence. They are also engineered or optimized to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. Because the sequences are novel, they facilitate detection using nucleic acid-based assays.

As used herein, “PCCB” refers to the beta subunit of human propionyl-CoA carboxylase, and “Pccb” refers to the beta subunit of mouse propionyl-CoA carboxylase. Propionyl-CoA carboxylase (PCC) catalyzes the carboxylation of propionyl-CoA to D-methylmalonyl-CoA which is a metabolic precursor to succinyl-CoA, a component of the citric acid cycle or tricarboxylic acid cycle (TCA). The genes encoding the alpha and beta subunits of naturally occurring human propionyl-CoA carboxylase gene are referred to as PCCB or PCCB, respectively. The synthetic polynucleotide encoding the beta subunit of PCC is known as synPCCB.

Naturally occurring human propionyl-CoA carboxylase is referred to as PCC, while synthetic PCC is designated as synPCC, even though the two are identical at the amino acid level.

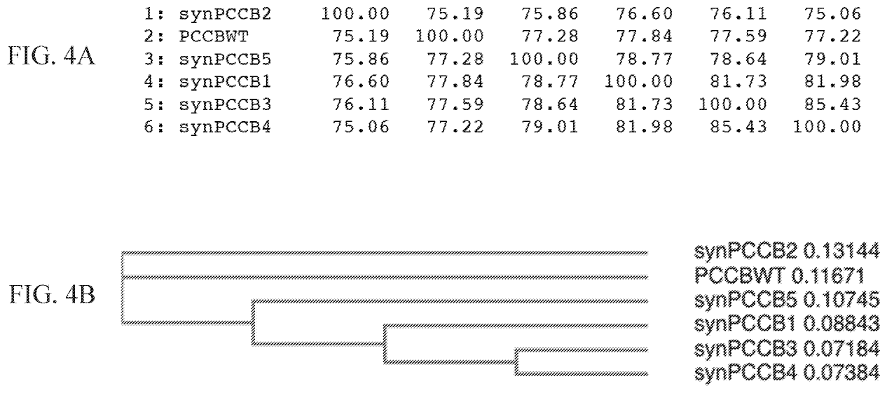

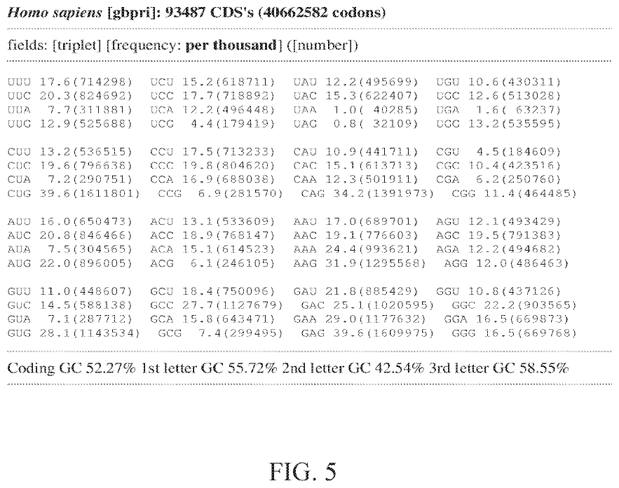

“Codon optimization” refers to the process of altering a naturally occurring polynucleotide sequence to enhance expression in the target organism, e.g., humans. In the subject application, the human PCCB gene has been altered to replace codons that occur less frequently in human genes with those that occur more frequently and/or with codons that are frequently found in highly expressed human genes, see FIG. 4.

As used herein, “determining”, “determination”, “detecting”, or the like are used interchangeably herein and refer to the detecting or quantitation (measurement) of a molecule using any suitable method, including immunohistochemistry, fluorescence, chemiluminescence, radioactive labeling, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and its variations refer to the identification or observation of the presence of a molecule in a biological sample, and/or to the measurement of the molecule's value.

As used herein, a “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In certain embodiments, it may be preferable to include isotonic agents, for example, sugars, polyalcohols such as mannitol, sorbitol, or sodium chloride in the composition.

A “therapeutically effective amount” refers to an amount effective, at dosages and for periods of time necessary, to achieve the desired therapeutic result. A therapeutically effective amount of a vector comprising the synthetic polynucleotide of the invention may vary according to factors such as the disease state, age, sex, and weight of the individual, and the ability of the vector to elicit a desired response in the individual. A therapeutically effective amount is also one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the synthetic polynucleotide or a fragment thereof according to the invention calculated to produce the desired therapeutic effect in association with a pharmaceutical carrier.

Table 1 below provides abbreviations used in the present application

TABLE 1

| Abbreviation | Long-form meaning |
|---|---|
| PA | Propionic acidemia |
| PCCB | beta subunit of propionyl-CoA carboxylase |
| PCC | propionyl-CoA carboxylase |
| PCCB (italicized) | Gene encoding beta subunit of propionyl-CoA |
| | carboxylase |
| AAV | adeno-associated virus |
| rAAV | recombinant adeno-associated virus |
| ITR | inverted terminal repeat |
| TEE | translational enhancer element |
| HPRE | Hepatitis B virus posttranscriptional |
| | regulatory element |
| EF1α | elongation factor 1 alpha promoter |
| EF1L | elongation factor 1 alpha promoter, long |
| hAAT | human alpha 1-antitrypsin promoter |
| CBA | chicken β-actin |
| CMV | cytomegalovirus immediate early gene promoter |
| TTR | Transthyretin promoter |
| TBG | thyroxine binding globulin promoter |
| A1AT | alpha-1 anti-trypsin promoter |
| CAG | CMV early enhancer element promoter construct |
| ApoE | apolipoprotein E enhancer |
| CMV | cytomegalovirus (CMV) immediate early gene |
| | enhancer |
| enTTR | transthyretin enhancer |
| rHBB | rabbit hemoglobin subunit beta intron |
| BGH | Bovine growth hormone |
| RBG | Rabbit beta-globin poly adenylation sequence |
| WPRE | Woodchuck Hepatitis Virus Posttranscriptional |
| | regulatory Element |

### Additional Embodiments of the Invention

### The Synthetic Polynucleotide

In one embodiment of the invention, codon optimization was employed to create six highly active and synthetic PCCB alleles designated PCCB1-5. This method involves determining the relative frequency of a codon in the protein-encoding genes in the human genome. For example, isoleucine can be encoded by AUU, AUC, or AUA, but in the human genome, AUC (47%), AUU (36%), and AUA (17%) are variably used to encode isoleucine in proteins. Therefore, in the proper sequence context, AUA would be changed to AUC to allow this codon to be more efficiently translated in human cells. FIG. 4 presents the codon usage statistics for a large fraction of human protein-encoding genes and serves as the basis for changing the codons throughout the PCCB cDNA.

Thus, the invention comprises synthetic polynucleotides encoding propionyl-CoA carboxylase subunit beta (PCCB) selected from the group consisting of SEQ ID NOs: 2-6 and a polynucleotide sequence having at least about 80% identity thereto. For those polynucleotides having at least about 80% identity to SEQ ID NOs: 2-6, in additional embodiments, they have at least 85%, at least 90%, at least 95%, at least 97%, at least 98%, or at least 99% identity.

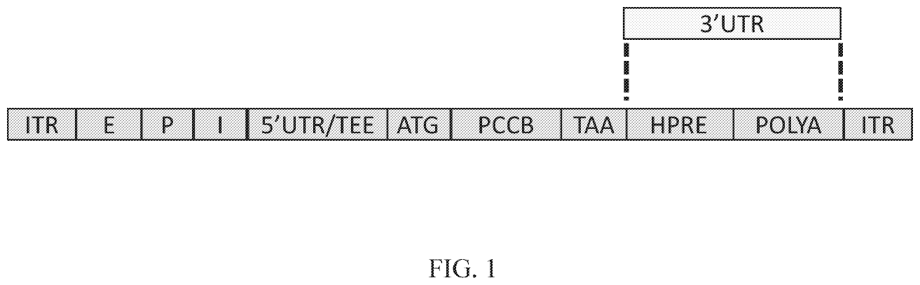

In one embodiment, the subject synthetic polynucleotide encodes a polypeptide with 100% identity to the naturally occurring human PCC protein. FIG. 1A presents the percent sequence identity of different PCCB alleles versus wild type PCCB, and each other, showing that all the synPCCB sequences (SEQ ID NOs: 2-6) differ from the wild type PCCB gene (SEQ ID NO: 1) by >18% at the nucleotide level, and similarly, as seen in FIG. 1B, widely diverge from each other based on phylogeny. Beyond sequence identity and alignments, the distinct features of the synPCCB sequences are further characterized using a phylogenetic analysis and presented in FIG. 1B where distinct grouping is apparent. FIG. 1B shows the characterization of distinct feature of the synPCCB sequences (SEQ ID NOs: 2-6) and the wild type PCCB gene (SEQ ID NO: 1) using a phylogenetic analysis where distinct grouping is apparent.

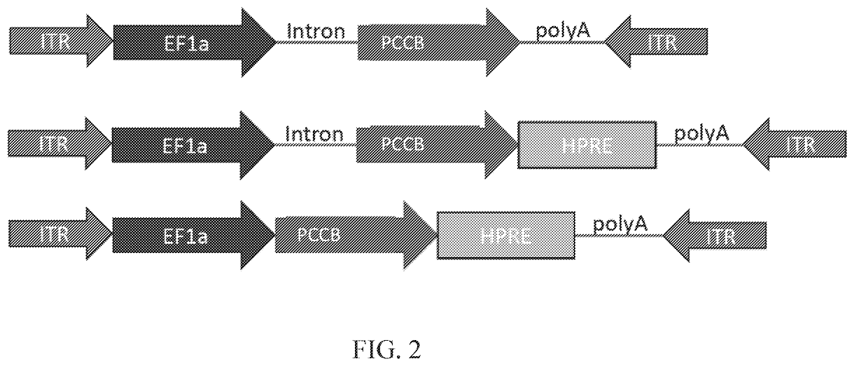

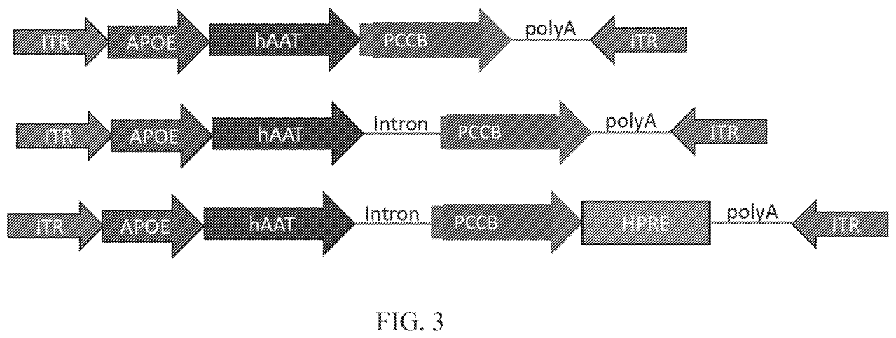

As can be seen in FIG. 2, the synPCCB1 and synPCCB2 constructs direct the expression of a greater amount of PCCB than the wild-type control. The promising expression data from both constructs led to the production AAV9-EF1a-synPCCB1 which was delivered to mice with propionic acidemia (Pccb−/−) by intrahepatic injection at birth. As presented in FIG. 3A, >50% of the Pccb−/− mice that received the AAV lived to 30 days, and further had an improved clinical appearance, as compared to the untreated Pccb−/− mice which had 100% mortality in early life. A substantial reduction in the disease related metabolite methylcitrate accompanied the rescue as seen in FIG. 3B.

Table 2 lists the DNA sequence of PCCB and the 5 alleles that were synthesized using different algorithms and expert adjustments to the sequence (synPCCB1-5).

TABLE 2
Sequences of wild-type and codon-optimized (or syn) PCCB alleles

| PCCB Allele | Sequences |
|---|---|
| wtPCCB | SEQ ID NO: 1 |
| synPCCB1 | SEQ ID NO: 2 |
| synPCCB2 | SEQ ID NO: 3 |
| synPCCB3 | SEQ ID NO: 4 |
| synPCCB4 | SEQ ID NO: 5 |
| synPCCB5 | SEQ ID NO: 6 |

Table 3 presents a multiple sequence alignment which clearly shows that the divergence in the sequences is distributed throughout.

TABLE 3
Sequence alignment of the synthetic PCCB alleles compared to each other and the
wild type PCCB sequence using CLUSTAL multiple sequence alignment by MUSCLE (3.8)

| | | |
|---|---|---|
| synPCCB2 | ATGGCTGCCGCCCTTAGAGTTGCCGCAGTTGGCGCGAGGCTCTCCGTGCTTGCTTCTGGC | 60 |
| PCCBWT | ATGGCGGCGGCATTACGGGTGGCGGCGGTCGGGGCAAGGCTCAGCGTTCTGGCGAGCGGT | 60 |
| synPCCB5 | ATGGCAGCTGCCCTTAGGGTGGCAGCGGTGGGCGCTAGGCTCAGTGTGCTCGCATCCGGC | 60 |
| synPCCB1 | ATGGCAGCTGCCCTTAGAGTCGCCGCAGTCGGAGCTAGACTGAGCGTGCTCGCGTCAGGT | 60 |
| synPCCB3 | ATGGCTGCCGCTCTGAGAGTCGCCGCTGTCGGGGCTAGGCTGAGTGTGCTGGCTTCTGGG | 60 |
| synPCCB4 | ATGGCTGCTGCTCTGAGAGTGGCTGCTGTGGGAGCTAGACTGTCTGTGCTGGCCTCTGGA | 60 |
| | ***** ** ** * * ** ** ** ** ** ** ** ** ** ** ** ** | |
| synPCCB2 | TTGCGCGCTGCTGTGCGCTCCCTGTGCAGCCAGGCGACCTCCGTCAATGAGCGGATTGAG | 120 |
| PCCBWT | CTCCGCGCCGCGGTCCGCAGCCTTTGCAGCCAGGCCACCTCTGTTAACGAACGCATCGAA | 120 |
| synPCCB5 | CTGAGAGCCGCTGTGCGTTCACTCTGCTCTCAGGCAACTAGCGTCAATGAGCGAATTGAG | 120 |
| synPCCB1 | CTGCGAGCCGCTGTGAGGAGCCTGTGCAGCCAGGCCACTTCCGTGAACGAACGGATTGAG | 120 |
| synPCCB3 | CTGCGCGCCGCCGTGAGGTCCCTGTGCTCCCAGGCCACCTCTGTGAACGAGAGGATCGAG | 120 |
| synPCCB4 | CTGAGAGCCGCCGTCAGATCTCTGTGTAGCCAGGCCACCAGCGTGAACGAGCGGATCGAG | 120 |
| | * * ** ** ** * ** ** ***** ** ** ** ** * ** ** | |
| synPCCB2 | AATAAGAGACGCACTGCCCTCTTGGGCGGTGGCCAGAGACGAATCGACGCCCAACACAAG | 180 |
| PCCBWT | AACAAGCGCCGGACCGCGCTGCTGGGAGGGGGCCAACGCCGTATTGACGCGCAGCACAAG | 180 |
| synPCCB5 | AACAAGCGCCGTACGGCATTGCTTGGAGGAGGTCAGCGACGTATTGATGCGCAGCACAAA | 180 |
| synPCCB1 | AACAAGAGGCGGACTGCCCTGCTGGGAGGAGGACAGCGGCGCATCGATGCACAGCACAAA | 180 |
| synPCCB3 | AATAAGCGGAGAACAGCCCTGCTGGGAGGAGGACAGAGGAGGATCGACGCACAGCACAAG | 180 |
| synPCCB4 | AACAAGCGGAGAACAGCCCTTCTTGGCGGCGGACAGAGAAGAATCGATGCCCAGCACAAG | 180 |
| | ** *** * * ** ** * * ** ** ** ** * * ** ** ** ** ***** | |
| synPCCB2 | CGCGGGAAACTCACAGCTCGGGAGAGAATTTCACTCCTTCTCGATCCAGGGTCTTTTGTA | 240 |
| PCCBWT | CGAGGAAAGCTAACAGCCAGGGAGAGGATCAGTCTCTTGCTGGACCCTGGCAGCTTTGTT | 240 |
| synPCCB5 | CGGGGAAAGCTGACTGCCAGAGAAAGGATCTCTCTGCTGTTGGATCCCGGAAGCTTTGTG | 240 |
| synPCCB1 | CGCGGCAAGCTGACTGCCCGCGAACGGATCTCCCTTCTCCTGGATCCCGGCTCCTTCGTG | 240 |
| synPCCB3 | AGGGGCAAGCTGACCGCAAGGGAGAGAATCTCTCTGCTGCTGGACCCAGGCAGCTTCGTG | 240 |
| synPCCB4 | CGGGGCAAGCTGACAGCCAGAGAGAGAATCAGCCTGCTGCTGGACCCTGGCAGCTTCGTG | 240 |
| | * ** ** ** ** ** * ** * ** ** * * ** ** ** ** ** | |
| synPCCB2 | GAGTCAGATATGTTTGTGGAACACCGATGCGCGGATTTTGGAATGGCGGCTGATAAGAAC | 300 |
| PCCBWT | GAGAGCGACATGTTTGTGGAACACAGATGTGCAGATTTTGGAATGGCTGCTGATAAGAAT | 300 |
| synPCCB5 | GAGTCTGACATGTTCGTGGAGCATCGATGTGCCGATTTTGGCATGGCAGCTGACAAGAAC | 300 |
| synPCCB1 | GAATCCGATATGTTTGTGGAACACCGCTGCGCCGACTTTGGAATGGCAGCCGACAAGAAC | 300 |
| synPCCB3 | GAGTCCGATATGTTTGTGGAGCACAGGTGCGCAGACTTCGGAATGGCAGCCGATAAGAAC | 300 |
| synPCCB4 | GAAAGCGACATGTTCGTGGAACACAGATGCGCCGACTTTGGCATGGCCGCCGACAAGAAC | 300 |
| | ** ** ***** ***** ** * ** ** ** ** ** ***** ** ** ***** | |
| synPCCB2 | AAATTCCCAGGGGACAGTGTCGTAACAGGTCGCGGGAGAATCAATGGCCGATTGGTCTAC | 360 |
| PCCBWT | AAGTTTCCTGGAGACAGCGTGGTCACTGGACGAGGCCGAATCAATGGAAGATTGGTTTAT | 360 |
| synPCCB5 | AAGTTCCCTGGCGACAGCGTGGTTACAGGCAGAGGGAGGATTAACGGCAGGCTCGTCTAC | 360 |
| synPCCB1 | AAGTTCCCTGGAGACTCCGTGGTGACCGGTCGGGGACGCATTAATGGCCGCCTGGTGTAC | 360 |
| synPCCB3 | AAGTTTCCAGGCGACTCCGTGGTGACCGGAAGGGGACGCATCAATGGCAGACTGGTGTAC | 360 |
| synPCCB4 | AAGTTCCCCGGCGATTCTGTGGTCACCGGCAGAGGCAGAATCAACGGCAGACTGGTGTAC | 360 |
| | ** ** ** ** ** ** ** ** ** * ** * ** ** ** * * ** ** | |
| synPCCB2 | GTTTTCTCCCAAGATTTTACGGTCTTCGGGGGATCACTGTCCGGAGCCCACGCTCAAAAG | 420 |
| PCCBWT | GTCTTCAGTCAGGATTTTACAGTTTTTGGAGGCAGTCTGTCAGGAGCACATGCCCAAAAG | 420 |
| synPCCB5 | GTCTTTAGCCAGGATTTCACCGTCTTTGGCGGGAGTTTGTCCGGAGCCCATGCCCAGAAA | 420 |
| synPCCB1 | GTGTTCAGCCAGGACTTCACTGTGTTCGGCGGAAGCCTGAGCGGAGCCCACGCTCAGAAG | 420 |
| synPCCB3 | GTGTTCTCTCAGGATTTCACAGTGTTTGGAGGCTCTCTGAGCGGAGCACACGCACAGAAG | 420 |
| synPCCB4 | GTGTTCAGCCAGGACTTCACCGTGTTTGGCGGAAGCCTGTCTGGCGCCCACGCTCAGAAA | 420 |
| | ** ** ** ** ** ** ** ** ** ** ** ** ** ** ** ** ** | |
| synPCCB2 | ATATGTAAAATCATGGACCAAGCTATAACAGTGGGGGCCCCTGTTATTGGCTTGAATGAC | 480 |
| PCCBWT | ATCTGCAAAATCATGGACCAGGCCATAACGGTGGGGGCTCCAGTGATTGGGCTGAATGAC | 480 |
| synPCCB5 | ATCTGCAAGATCATGGATCAGGCAATCACAGTCGGAGCTCCTGTTATAGGACTGAACGAC | 480 |
| synPCCB1 | ATCTGCAAGATCATGGACCAGGCCATCACGGTCGGAGCTCCGGTCATCGGCCTCAACGAC | 480 |
| synPCCB3 | ATCTGTAAGATCATGGACCAGGCAATCACCGTGGGAGCACCTGTGATCGGCCTGAACGAT | 480 |
| synPCCB4 | ATCTGCAAGATCATGGACCAGGCCATCACCGTGGGAGCCCCTGTGATCGGCCTTAATGAT | 480 |
| | ** ** ** ******** ** ** ** ** ** ** ** ** ** ** ** * ** ** | |
| synPCCB2 | AGCGGTGGTGCGCGGATACAGGAAGGTGTTGAGTCTTTGGCTGGGTATGCTGACATTTTT | 540 |
| PCCBWT | TCTGGGGGAGCACGGATCCAAGAAGGAGTGGAGTCTTTGGCTGGCTATGCAGACATCTTT | 540 |
| synPCCB5 | TCAGGCGGCGCTAGAATTCAGGAAGGTGTCGAGTCTCTGGCGGGGTATGCCGACATCTTT | 540 |
| synPCCB1 | TCCGGTGGAGCACGGATCCAAGAGGGAGTGGAATCACTGGCCGGCTATGCGGACATTTTC | 540 |
| synPCCB3 | TCCGGAGGAGCAAGGATCCAGGAGGGAGTGGAGTCTCTGGCCGGCTATGCCGATATCTTT | 540 |
| synPCCB4 | TCTGGCGGAGCCCGGATCCAAGAGGGCGTTGAATCTCTGGCCGGCTACGCCGACATCTTC | 540 |
| | ** ** ** * ** ** ** ** ** ** ** **** ** ** ** ** ** ** | |
| synPCCB2 | CTCCGGAATGTTACAGCATCTGGTGTTATACCCCAGATTTCTCTGATAATGGGCCCATGC | 600 |
| PCCBWT | CTGAGGAATGTTACGGCATCCGGAGTCATCCCTCAGATTTCTCTGATCATGGGCCCATGT | 600 |
| synPCCB5 | CTGCGGAATGTGACTGCTTCAGGTGTGATACCTCAAATCAGTCTGATCATGGGGCCATGC | 600 |
| synPCCB1 | CTGAGAAACGTGACTGCCTCCGGGGTGATCCCTCAGATCTCCCTGATCATGGGGCCCTGT | 600 |
| synPCCB3 | CTGCGCAATGTGACAGCCTCTGGCGTGATCCCACAGATCAGCCTGATCATGGGACCATGC | 600 |
| synPCCB4 | CTGAGAAATGTGACCGCCAGCGGCGTGATCCCTCAGATCAGCCTGATCATGGGACCTTGT | 600 |
| | ** * ** ** ** ** ** ** ** ** ** ** ***** ***** ** ** | |
| synPCCB2 | GCGGGGGGGGCTGTCTATAGCCCGGCGCTGACCGATTTCACATTCATGGTCAAGGATACG | 660 |
| PCCBWT | GCTGGTGGGGCCGTCTACTCCCCAGCCCTAACAGACTTCACGTTCATGGTAAAGGACACC | 660 |
| synPCCB5 | GCAGGAGGAGCCGTTTACTCTCCGGCACTCACCGACTTTACCTTCATGGTGAAAGACACC | 660 |
| synPCCB1 | GCTGGAGGAGCCGTGTACTCACCGGCCCTGACCGATTTCACCTTCATGGTCAAGGATACC | 660 |
| synPCCB3 | GCAGGAGGAGCCGTGTACTCCCCTGCCCTGACCGACTTCACCTTCATGGTGAAGGATACC | 660 |
| synPCCB4 | GCAGGCGGAGCCGTGTACTCTCCTGCTCTGACCGACTTCACCTTCATGGTCAAGGACACC | 660 |
| | ** ** ** ** ** ** ** ** ** ** ** ** ** ******** ** ** ** | |
| synPCCB2 | TCCTATCTCTTTATTACTGGTCCCGACGTTGTCAAGTCTGTGACTAATGAAGACGTCACT | 720 |
| PCCBWT | TCCTACCTGTTCATCACTGGCCCTGATGTTGTGAAGTCTGTCACCAATGAGGATGTTACC | 720 |
| synPCCB5 | TCCTACCTGTTCATCACAGGGCCAGATGTGGTCAAATCCGTGACCAACGAGGACGTTACA | 720 |
| synPCCB1 | TCGTACCTCTTCATTACTGGCCCAGACGTGGTCAAGTCCGTGACCAACGAGGACGTGACC | 720 |
| synPCCB3 | AGCTATCTGTTCATCACAGGCCCAGACGTGGTGAAGTCCGTGACCAACGAGGATGTGACA | 720 |
| synPCCB4 | AGCTACCTGTTCATCACTGGCCCCGACGTGGTCAAGAGCGTGACCAATGAGGACGTGACC | 720 |
| | ** ** ** ** ** ** ** ** ** ** ** ** ** ** ** ** ** ** | |
| synPCCB2 | CAAGAAGAGTTGGGTGGCGCTAAGACTCACACTACCATGAGTGGCGTGGCTCATCGAGCA | 780 |
| PCCBWT | CAGGAGGAGCTCGGTGGTGCCAAGACCCACACCACCATGTCAGGTGTGGCCCACAGAGCT | 780 |
| synPCCB5 | CAGGAAGAGCTGGGAGGCGCAAAGACGCACACAACCATGAGTGGAGTGGCACACAGAGCC | 780 |
| synPCCB1 | CAGGAGGAGTTGGGAGGCGCCAAGACACACACCACCATGAGCGGAGTCGCCCATCGGGCG | 780 |
| synPCCB3 | CAGGAGGAGCTGGGAGGAGCAAAGACCCACACCACAATGAGCGGAGTGGCACACAGGGCC | 780 |
| synPCCB4 | CAAGAGGAACTCGGCGGAGCCAAGACACACACCACAATGAGCGGAGTGGCCCACAGAGCC | 780 |
| | ** ** ** * ** ** ** ***** ***** ** *** ** ** ** ** * ** | |
| synPCCB2 | TTTGAGAACGACGTTGATGCACTTTGTAACCTTCGAGATTTTTTCAATTATCTTCCGCTC | 840 |
| PCCBWT | TTTGAAAATGATGTTGATGCCTTGTGTAATCTCCGGGATTTCTTCAACTACCTGCCCCTG | 840 |
| synPCCB5 | TTCGAAAACGACGTCGATGCCCTGTGCAATCTTAGGGATTTCTTCAATTACCTCCCTCTG | 840 |
| synPCCB1 | TTCGAGAACGATGTCGACGCCCTGTGCAATCTGCGGGATTTCTTTAACTACCTCCCGCTG | 840 |
| synPCCB3 | TTTGAGAATGACGTGGATGCCCTGTGCAACCTGAGAGACTTCTTTAATTACCTGCCTCTG | 840 |
| synPCCB4 | TTCGAGAACGATGTGGACGCCCTGTGCAACCTGCGGGACTTCTTCAACTACCTGCCTCTG | 840 |
| | ** ** ** ** ** ** ** * ** ** ** * ** ** ** ** ** ** ** ** | |
| synPCCB2 | TCTTCACAAGATCCCGCTCCAGTTAGAGAGTGCCATGATCCAAGTGATAGGCTGGTACCC | 900 |
| PCCBWT | AGCAGTCAGGACCCGGCTCCCGTCCGTGAGTGCCACGATCCCAGTGACCGTCTGGTTCCT | 900 |
| synPCCB5 | TCATCCCAAGATCCCGCACCAGTGAGAGAGTGTCACGACCCCTCAGACCGCTTGGTACCC | 900 |
| synPCCB1 | TCCAGCCAAGACCCTGCACCTGTGCGGGAATGCCACGACCCAAGCGACAGGCTGGTGCCG | 900 |
| synPCCB3 | AGCTCCCAGGATCCAGCACCTGTGAGGGAGTGTCACGACCCATCCGATCGCCTGGTGCCT | 900 |
| synPCCB4 | AGCAGCCAGGATCCAGCTCCTGTGCGGGAATGTCACGACCCCTCTGATAGACTGGTGCCC | 900 |
| | ** ** ** ** ** ** * ** ** ** ** ** ** * **** ** | |
| synPCCB2 | GAACTCGATACCATAGTTCCTTTGGAATCTACTAAAGCGTACAACATGGTCGACATAATC | 960 |
| PCCBWT | GAGCTTGACACAATTGTCCCTTTGGAATCAACCAAAGCCTACAACATGGTGGACATCATA | 960 |
| synPCCB5 | GAACTGGATACCATTGTCCCACTGGAGAGCACCAAAGCCTACAATATGGTTGACATTATA | 960 |
| synPCCB1 | GAACTCGACACCATTGTGCCTCTGGAATCGACCAAGGCGTACAACATGGTCGATATCATC | 960 |
| synPCCB3 | GAGCTGGACACCATCGTGCCACTGGAGTCTACAAAGGCCTACAACATGGTGGACATCATC | 960 |
| synPCCB4 | GAGCTGGATACCATCGTGCCTCTGGAAAGCACCAAGGCCTACAACATGGTGGACATCATC | 960 |
| | ** ** ** ** ** ** ** **** ** ** ** ***** ***** ** ** ** | |
| synPCCB2 | CACAGTGTAGTGGACGAAAGGGAATTTTTTGAGATTATGCCAAATTACGCGAAGAACATT | 1020 |
| PCCBWT | CACTCTGTTGTTGATGAGCGTGAATTTTTTGAGATCATGCCCAATTATGCCAAGAACATC | 1020 |
| synPCCB5 | CACAGCGTCGTTGATGAACGAGAGTTCTTTGAGATAATGCCCAACTACGCCAAGAACATC | 1020 |
| synPCCB1 | CACTCTGTGGTGGATGAACGCGAATTCTTCGAGATCATGCCGAACTACGCCAAGAATATT | 1020 |
| synPCCB3 | CACAGCGTGGTGGATGAGAGGGAGTTCTTTGAGATCATGCCCAACTATGCCAAGAATATC | 1020 |
| synPCCB4 | CACAGCGTGGTGGACGAGCGCGAGTTCTTCGAGATCATGCCCAACTACGCCAAGAACATC | 1020 |
| | *** ** ** ** ** * ** ** ** ***** ***** ** ** ** ***** ** | |
| synPCCB2 | ATAGTGGGCTTTGCGCGGATGAACGGGCGAACCGTAGGCATAGTAGGGAACCAACCGAAA | 1080 |
| PCCBWT | ATTGTTGGTTTTGCAAGAATGAATGGGAGGACTGTTGGAATTGTTGGCAACCAACCTAAG | 1080 |
| synPCCB5 | ATTGTGGGCTTTGCTAGGATGAATGGCCGGACTGTTGGTATAGTGGGCAATCAGCCTAAG | 1080 |
| synPCCB1 | ATCGTGGGATTTGCTCGGATGAACGGACGCACTGTGGGCATTGTCGGCAACCAGCCCAAG | 1080 |
| synPCCB3 | ATCGTGGGCTTCGCCCGGATGAACGGCAGAACCGTGGGCATCGTGGGCAATCAGCCTAAG | 1080 |
| synPCCB4 | ATCGTGGGCTTCGCCCGGATGAACGGCAGAACAGTGGGCATCGTGGGAAACCAGCCTAAG | 1080 |
| | ** ** ** ** ** * ***** ** * ** ** ** ** ** ** ** ** ** ** | |
| synPCCB2 | GTGGCTTCAGGTTGCCTGGATATCAATAGCAGCGTGAAGGGAGCCAGATTTGTGAGGTTT | 1140 |
| PCCBWT | GTGGCCTCAGGATGCTTGGATATTAATTCATCTGTGAAAGGGGCTCGTTTTGTCAGATTC | 1140 |
| synPCCB5 | GTAGCGTCCGGGTGTCTGGACATAAATTCCAGTGTGAAGGGTGCCCGGTTCGTACGCTTC | 1140 |
| synPCCB1 | GTCGCCTCCGGATGCCTGGACATTAACTCCTCCGTGAAGGGCGCGAGGTTTGTGCGCTTC | 1140 |
| synPCCB3 | GTGGCCAGCGGCTGCCTGGATATCAACTCTAGCGTGAAGGGCGCCAGGTTCGTGCGCTTT | 1140 |
| synPCCB4 | GTGGCAAGCGGCTGCCTGGACATCAACAGCTCTGTGAAAGGCGCCAGATTCGTGCGGTTC | 1140 |
| | ** ** ** ** **** ** ** ***** ** ** * ** ** * ** | |
| synPCCB2 | TGTGACGCTTTCAATATCCCTCTTATAACCTTTGTCGATGTCCCTGGGTTCCTGCCTGGG | 1200 |
| PCCBWT | TGTGATGCATTCAATATTCCACTCATCACTTTTGTTGATGTCCCTGGCTTTCTACCTGGC | 1200 |
| synPCCB5 | TGCGATGCGTTTAACATTCCGCTTATCACATTCGTGGATGTGCCAGGGTTTCTGCCTGGG | 1200 |
| synPCCB1 | TGCGACGCCTTTAACATTCCCCTGATTACCTTCGTCGATGTCCCCGGATTCCTGCCTGGC | 1200 |
| synPCCB3 | TGTGACGCCTTCAATATCCCACTGATCACCTTCGTGGATGTGCCTGGCTTTCTGCCAGGA | 1200 |
| synPCCB4 | TGCGACGCCTTCAACATCCCTCTGATCACCTTCGTGGACGTGCCCGGCTTTCTGCCTGGA | 1200 |
| | ** ** ** ** ** ** ** ** ** ** ** ** ** ** ** ** ** ** ** ** | |
| synPCCB2 | ACTGCGCAGGAGTATGGCGGTATAATACGACATGGCGCTAAGCTGCTTTACGCATTCGCG | 1260 |
| PCCBWT | ACAGCACAGGAATACGGGGGCATCATCCGGCATGGTGCCAAGCTTCTCTACGCATTTGCT | 1260 |
| synPCCB5 | ACTGCCCAGGAATATGGTGGGATTATCCGGCATGGGGCCAAACTGTTGTATGCCTTCGCC | 1260 |
| synPCCB1 | ACCGCTCAGGAATACGGGGGCATCATCCGCCATGGCGCCAAGCTGCTCTATGCCTTCGCC | 1260 |
| synPCCB3 | ACAGCACAGGAGTACGGAGGAATCATCAGGCACGGAGCAAAGCTGCTGTATGCCTTTGCC | 1260 |
| synPCCB4 | ACAGCCCAAGAGTATGGCGGCATCATTAGACACGGCGCCAAGCTGCTGTACGCCTTTGCC | 1260 |
| | ** ** ** ** ** ** ** ** ** * ** ** ** ** ** * ** ** ** ** | |
| synPCCB2 | GAAGCGACAGTTCCTAAAGTTACTGTGATTACGCGCAAAGCATATGGTGGCGCCTACGAT | 1320 |
| PCCBWT | GAGGCAACTGTACCCAAAGTCACAGTCATCACCAGGAAGGCCTATGGAGGTGCCTATGAT | 1320 |
| synPCCB5 | GAAGCGACCGTACCCAAGGTTACGGTGATTACACGCAAAGCCTATGGCGGTGCCTATGAT | 1320 |
| synPCCB1 | GAAGCCACCGTGCCAAAAGTGACTGTGATCACCCGGAAGGCTTACGGCGGTGCCTATGAC | 1320 |
| synPCCB3 | GAGGCCACAGTGCCCAAGGTGACCGTGATCACAAGAAAGGCCTACGGCGGCGCCTATGAC | 1320 |
| synPCCB4 | GAAGCCACCGTGCCTAAAGTGACCGTGATCACCAGAAAGGCCTATGGCGGCGCTTACGAC | 1320 |
| | ** ** ** ** ** ** ** ** ** ** ** * ** ** ** ** ** ** ** ** | |
| synPCCB2 | GTCATGTCCAGTAAGCATCTTTGCGGAGACACTAATTATGCATGGCCGACAGCAGAGATC | 1380 |
| PCCBWT | GTCATGAGCTCTAAGCACCTTTGTGGTGATACCAACTATGCCTGGCCCACCGCAGAGATT | 1380 |
| synPCCB5 | GTCATGAGCAGCAAGCACCTTTGTGGCGACACAAACTATGCATGGCCTACTGCTGAAATC | 1380 |
| synPCCB1 | GTGATGTCGTCGAAGCACTTGTGTGGCGACACTAACTATGCCTGGCCCACTGCCGAGATC | 1380 |
| synPCCB3 | GTGATGTCCTCTAAGCACCTGTGCGGCGATACCAATTACGCCTGGCCTACAGCCGAGATC | 1380 |
| synPCCB4 | GTGATGAGCAGCAAGCACCTGTGCGGCGACACCAATTACGCCTGGCCTACAGCCGAGATC | 1380 |
| | ** *** ***** * ** ** ** ** ** ** ** ***** ** ** ** ** | |
| synPCCB2 | GCTGTGATGGGCGCGAAGGGAGCGGTCGAGATAATATTCAAAGGACATGAAAATGTCGAA | 1440 |
| PCCBWT | GCAGTCATGGGAGCAAAGGGCGCTGTGGAGATCATCTTCAAAGGGCATGAGAATGTGGAA | 1440 |
| synPCCB5 | GCTGTCATGGGAGCTAAAGGGGCTGTAGAGATTATCTTTAAAGGTCATGAAAACGTGGAA | 1440 |
| synPCCB1 | GCTGTGATGGGAGCTAAAGGGGCCGTGGAGATCATCTTCAAGGGGCATGAGAACGTGGAA | 1440 |
| synPCCB3 | GCCGTGATGGGAGCAAAGGGAGCAGTGGAGATCATCTTCAAGGGACACGAGAACGTGGAG | 1440 |
| synPCCB4 | GCCGTGATGGGAGCTAAAGGCGCCGTGGAAATCATCTTCAAGGGCCACGAGAACGTGGAA | 1440 |
| | ** ** ***** ** ** ** ** ** ** ** ** ** ** ** ** ** ** ** ** | |
| synPCCB2 | GCGGCTCAGGCAGAGTACATAGAGAAATTCGCAAACCCGTTCCCCGCTGCCGTGCGAGGA | 1500 |
| PCCBWT | GCTGCTCAGGCAGAGTACATCGAGAAGTTTGCCAACCCTTTCCCTGCAGCAGTGCGAGGG | 1500 |
| synPCCB5 | GCCGCTCAAGCCGAGTACATCGAGAAGTTTGCTAATCCCTTCCCAGCTGCCGTTAGAGGC | 1500 |
| synPCCB1 | GCCGCCCAAGCGGAATACATTGAGAAGTTCGCGAACCCCTTCCCTGCTGCCGTGAGAGGC | 1500 |
| synPCCB3 | GCAGCACAGGCCGAGTATATCGAGAAGTTCGCAAATCCATTTCCAGCAGCCGTGCGGGGA | 1500 |
| synPCCB4 | GCCGCTCAGGCCGAGTACATCGAGAAGTTCGCCAATCCTTTTCCTGCCGCCGTGCGGGGC | 1500 |
| | ** ** ** ** ** ** ** ***** ** ** ** ** ** ** ** ** ** * ** | |
| synPCCB2 | TTCGTAGATGACATTATTCAGCCTAGCAGCACCCGAGCTAGGATCTGCTGTGATCTTGAT | 1560 |
| PCCBWT | TTTGTGGATGACATCATCCAACCTTCTTCCACACGTGCCCGAATCTGCTGTGACCTGGAT | 1560 |
| synPCCB5 | TTTGTGGACGACATTATCCAGCCGTCTAGCACTCGCGCTCGGATCTGCTGTGACCTGGAT | 1560 |
| synPCCB1 | TTCGTGGACGACATCATCCAGCCGAGCTCGACCAGAGCGAGAATCTGCTGCGACCTCGAC | 1560 |
| synPCCB3 | TTTGTGGACGATATCATCCAGCCCAGCTCCACCCGGGCCAGAATCTGCTGTGACCTGGAC | 1560 |
| synPCCB4 | TTTGTGGACGATATCATTCAGCCCAGCAGCACCCGGGCCAGAATCTGCTGTGATCTGGAT | 1560 |
| | ** ** ** ** ** ** ** ** ** * ** * ******** ** ** ** | |
| synPCCB2 | GTGCTCGCTTCCAAGAAGGTACAGCGGCCCTGGCGGAAACATGCAAACATTCCCCTGTAA | 1620 |
| PCCBWT | GTCTTGGCCAGCAAGAAGGTACAACGTCCTTGGAGAAAACATGCAAATATTCCATTGTAA | 1620 |
| synPCCB5 | GTGCTGGCCAGCAAGAAGGTACAACGCCCATGGCGGAAACATGCCAATATTCCCCTCTAA | 1620 |
| synPCCB1 | GTCCTGGCGTCCAAGAAGGTCCAACGGCCTTGGAGGAAGCATGCAAACATCCCGCTGTAG | 1620 |
| synPCCB3 | GTGCTGGCTTCTAAAAAAGTGCAGAGACCTTGGCGGAAACATGCTAACATCCCCCTGTAA | 1620 |
| synPCCB4 | GTGCTGGCTAGCAAGAAGGTGCAGCGGCCTTGGAGAAAGCACGCCAACATTCCTCTGTAA | 1620 |
| | ** * ** ** ** ** ** * ** *** * ** ** ** ** ** ** * ** | |

Table 4 presents descriptions for SEQ ID NOs in the sequence listing for SEQ ID NOs: 7-41

TABLE 4

| Sequence ID | Description |
|---|---|
| 7 | PCCB |
| 8 | pAAV CBA synPCCB1 |
| 9 | pAAV EF1L synPCCB1 |
| 10 | pAAV EF1S synPCCB1 HPRE |
| 11 | pAAV hAAT synPCCB1 WPRE |
| 12 | APOE S |
| 13 | APOE L |
| 14 | HAAT S |
| 15 | HATT L |
| 16 | INTRON C |
| 17 | INTRON B |
| 18 | INTRON S |
| 19 | APOE S/HAAT S |
| 20 | APOE L/HATT L |
| 21 | APOE L/HATT L/INTRON B |
| 22 | AMBP enhancer |
| 23 | AMBP enhancer 2x |
| 24 | TBG |
| 25 | ENHANCED TBG |
| 26 | IRES |
| 27 | 5′UTR ALB |
| 28 | 5′UTR SERPINA 1 |
| 29 | 5′UTR SERPINA 3 |
| 30 | WPRE |
| 31 | HPRE |
| 32 | piggyBac 5′ inverted terminal repeat |
| 33 | piggyBac 3′ inverted terminal repeat |
| 34 | pAAV TBG synPCCB1 |
| 35 | pAAV HAAT synPCCB1 |
| 36 | pAAV 3′ALB synPCCB1 |
| 37 | pAAV 5′ ALB synPCCB1 |
| 38 | HAAT synPCCB1 lenti |
| 39 | EF1L synPCCB1 lenti |
| 40 | AAV EF1S PCCB HPRE KANA |

In another aspect, SEQ ID NOs:2-6 encode a PCC beta subunit that has 100% identity with the naturally occurring human PCC beta subunit protein, or that has at least 90% amino acid identity to the naturally occurring human PCC beta subunit protein. In a preferred embodiment, the polynucleotide encodes a PCC beta subunit protein that has at least 95% amino acid identity to naturally occurring human PCC beta subunit protein.

In one embodiment, a polypeptide according to the invention retains at least 90% of the naturally occurring human PCC protein function, i.e., the capacity to catalyze the carboxylation of propionyl-CoA to D-methylmalonyl-CoA. In another embodiment, the encoded PCC protein retains at least 95% of the naturally occurring human PCC protein function. This protein function can be measured, for example, via the efficacy to rescue a neonatal lethal phenotype in Pccb knock-out mice (FIG. 3A), the lowering of circulating metabolites including methylcitrate in a disease model of PA (FIG. 3B).

In some embodiments, the synthetic polynucleotide exhibits improved expression relative to the expression of naturally occurring human propionyl-CoA carboxylase beta polynucleotide sequence. The improved expression is due to the polynucleotide comprising codons that have been optimized relative to the naturally occurring human propionyl-CoA carboxylase beta polynucleotide sequence. In one aspect, the synthetic polynucleotide has at least about 25%, at least about 30%, at least about 35%, at least about 40%, at least about 45%, at least about 50%, at least about 55%, at least about 60%, at least about 65%, at least about 70%, at least about 75%, at least about 80% of less commonly used codons replaced with more commonly used codons. In additional embodiments, the polynucleotide has at least 85%, 90%, or 95% replacement of less commonly used codons with more commonly used codons, and demonstrate equivalent or enhanced expression of PCCB as compared to SEQ ID NO:1.

In some embodiments, the synthetic polynucleotide sequences of the invention preferably encode a polypeptide that retains at least about 80% of the enhanced PCC expression (as demonstrated by expression of the polynucleotide of SEQ ID NO:1 in an appropriate host.) In additional embodiments, the polypeptide retains at least 85%, 90%, or 95% or 100% of the enhanced expression observed with the polynucleotides of SEQ ID NOs:2-6.

In designing the synPCCB of the present invention, the following considerations were balanced. For example, the fewer changes that are made to the nucleotide sequence of SEQ ID NO:1, decreases the potential of altering the secondary structure of the sequence, which can have a significant impact on gene expression. The introduction of undesirable restriction sites is also reduced, facilitating the subcloning of PCCB into the plasmid expression vector. However, a greater number of changes to the nucleotide sequence of SEQ ID NO:1 allows for more convenient identification of the translated and expressed message, e.g. mRNA, in vivo. Additionally, greater number of changes to the nucleotide sequence of SEQ ID NO:1 provides for increased likelihood of greater expression. These considerations were balanced when arriving at SEQ ID NOs: 2-6. The polynucleotide sequences encoding synPCCB allow for increased expression of the synPCCB gene relative to naturally occurring human PCCB sequences. They are also engineered to have increased transcriptional, translational, and protein refolding efficacy. This engineering is accomplished by using human codon biases, evaluating GC, CpG, and negative GpC content, optimizing the interaction between the codon and anti-codon, and eliminating cryptic splicing sites and RNA instability motifs. Because the sequences are novel, they facilitate detection using nucleic acid-based assays.

PCCB has a total of 539 amino acids and synPCCB contains approximately 1617 codons corresponding to said amino acids. In SEQ ID NOs:2-6, codons are changed from that of the natural human PCCB, however, as described, SEQ ID NOs:2-6, despite changes from SEQ ID NO:1, codes for the amino acid sequence SEQ ID NO:7 for PCCB. Codons for SEQ ID NOs:2-6 are changed, in accordance with the equivalent amino acid positions of SEQ ID NO:7, as see in Table 3. In this embodiment, the amino acid sequence for natural human PCCB has been retained.

It can be appreciated that partial reversion of the designed synPCCB to codons that are found in PCCB can be expected to result in nucleic acid sequences that, when incorporated into appropriate vectors, can also exhibit the desirable properties of SEQ ID NOs:2-6, for example, such partial reversion variants can have equivalent expression of PCCB from a vector inserted into an appropriate host, as SEQ ID NOs:2-6. For example, the invention includes nucleic acids in which at least about 1 altered codon, at least about 2 altered codons, at least about 3, altered codons, at least about 4 altered codons, at least about 5 altered codons, at least about 6 altered codons, at least about 7 altered codons, at least about 8 altered codons, at least about 9 altered codons, at least about 10 altered codons, at least about 11 altered codons, at least about 12 altered codons, at least about 13 altered codons, at least about 14 altered codons, at least about 15 altered codons, at least about 16 altered codons, at least about 17 altered codons, at least about 18 altered codons, at least about 20 altered codons, at least about 25 altered codons, at least about 30 altered codons, at least about 35 altered codons, at least about 40 altered codons, at least about 50 altered codons, at least about 55 altered codons, at least about 60 altered codons, at least about 65 altered codons, at least about 70 altered codons, at least about 75 altered codons, at least about 80 altered codons, at least about 85 altered codons, at least about 90 altered codons, at least about 95 altered codons, at least about 100 altered codons, at least about 110 altered codons, at least about 120 altered codons, at least about 130 altered codons, at least about 130 altered codons, at least about 140 altered codons, at least about 150 altered codons, at least about 160 altered codons, at least about 170 altered codons, at least about 180 altered codons, at least about 190 altered codons, at least about 200 altered codons, at least about 220 altered codons, at least about 240 altered codons, at least about 260 altered codons, at least about 280 altered codons, at least about 300 altered codons, at least about 320 altered codons, at least about 340 altered codons, at least about 360 altered codons, at least about 380 altered codons, at least about 400 altered codons, at least about 420 altered codons, at least about 440 altered codons, at least about 460 altered codons, or at least about 480 of the altered codon positions in SEQ ID NOs:2-6 are reverted to native codons according to SEQ ID NO:1, and having equivalent expression to SEQ ID NO:1. Alternately, at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% of the altered codon positions in SEQ ID NOs:2-6 are reverted to native sequence according to SEQ ID NO:1, and having equivalent expression to SEQ ID NOs:2-6.

In some embodiments, polynucleotides of the present invention do not share 100% identity with SEQ ID NO:1. In other words, in some embodiments, polynucleotides having 100% identity with SEQ ID NO:1 are excluded from the embodiments of the present invention.

The synthetic polynucleotide can be composed of DNA and/or RNA or a modified nucleic acid, such as a peptide nucleic acid, and could be conjugated for improved biological properties.

### Therapy

In another aspect, the invention comprises a method of treating a disease or condition mediated by propionyl-CoA carboxylase. The disease or condition can, in one embodiment, be propionic acidemia (PA). This method comprises administering to a subject in need thereof a synthetic propionyl-CoA carboxylase polynucleotide construct comprising the synthetic polynucleotides (synPCCB) described herein. The PCC enzyme is processed after transcription, translation, and translocation into the mitochondrial inner space.

Enzyme replacement therapy consists of administration of the functional enzyme (propionyl-CoA carboxylase) to a subject in a manner so that the enzyme administered will catalyze the reactions in the body that the subject's own defective or deleted enzyme cannot. In enzyme therapy, the defective enzyme can be replaced in vivo or repaired in vitro using the synthetic polynucleotide according to the invention. The functional enzyme molecule can be isolated or produced in vitro, for example. Methods for producing recombinant enzymes in vitro are known in the art. In vitro enzyme expression systems include, without limitation, cell-based systems (bacterial (for example, Escherichia coli, Corynebacterium, Pseudomonas fluorescens), yeast (for example, Saccharomyces cerevisiae, Pichia Pastoris), insect cell (for example, Baculovirus-infected insect cells, non-lytic insect cell expression), and eukaryotic systems (for example, Leishmania)) and cell-free systems (using purified RNA polymerase, ribosomes, tRNA, ribonucleotides). Viral in vitro expression systems are likewise known in the art. The enzyme isolated or produced according to the above-iterated methods exhibits, in specific embodiments, 80%, 85%, 90%, 95%, 98%, 99%, or 100% homology to the naturally occurring (for example, human) propionyl-CoA carboxylase.

Gene therapy can involve in vivo gene therapy (direct introduction of the genetic material into the cell or body) or ex vivo gene transfer, which usually involves genetically altering cells prior to administration. In one aspect, genome editing, or genome editing with engineered nucleases (GEEN) may be performed with the synPCCB nucleotides of the present invention allowing synPCCB DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the nucleotides of the present invention including synPCCB, in combination with a CASP/CRISPR, ZFN, or TALEN can be used to engineer correction at the locus in a patient's cell either in vivo or ex vivo, then, in one embodiment, use that corrected cell, such as a fibroblast or lymphoblast, to create an iPS or other stem cell for use in cellular therapy.

### Administration/Delivery and Dosage Forms

Routes of delivery of a synthetic propionyl-CoA carboxylase (PCCB) polynucleotide according to the invention may include, without limitation, injection (systemic or at target site), for example, intradermal, subcutaneous, intravenous, intraperitoneal, intraocular, subretinal, renal artery, hepatic vein, intramuscular injection; physical, including ultrasound(-mediated transfection), electric field-induced molecular vibration, electroporation, transfection using laser irradiation, photochemical transfection, gene gun (particle bombardment); parenteral and oral (including inhalation aerosols and the like). Related methods include using genetically modified cells, antisense therapy, and RNA interference.

Vehicles for delivery of a synthetic propionyl-CoA carboxylase polynucleotide (synPCCB) according to the invention may include, without limitation, viral vectors (for example, AAV, adenovirus, baculovirus, retrovirus, lentivirus, foamy virus, herpes virus, Moloney murine leukemia virus, Vaccinia virus, and hepatitis virus) and non-viral vectors (for example, naked DNA, mini-circules, liposomes, ligand-polylysine-DNA complexes, nanoparticles, cationic polymers, including polycationic polymers such as dendrimers, synthetic peptide complexes, artificial chromosomes, and polydispersed polymers). Thus, dosage forms contemplated include injectables, aerosolized particles, capsules, and other oral dosage forms.

In certain embodiments, the vector used for gene therapy comprises an expression cassette. The expression cassette may, for example, consist of a promoter, the synthetic polynucleotide, and a polyadenylation signal. Viral promoters include, for example, the ubiquitous cytomegalovirus immediate early (CMV-IE) promoter, the chicken beta-actin (CBA) promoter, elongation factor 1 alpha (EF1a) promoter, the simian virus 40 (SV40) promoter, the Rous sarcoma virus long terminal repeat (RSV-LTR) promoter, the Moloney murine leukemia virus (MoMLV) LTR promoter, other retroviral LTR promoters, tissue restricted promoters to direct expression to the liver such as the thyroid binding globulin (TBG), human alpha-1 antitrypsin (hAAT), transthretinin (TTR); the muscle such as muscle creatine kinase (MCK), and desmin; and other housekeeping genes including pyruvate carboxylase (PC) and phosphoglycerol kinase (PGK). The promoters may vary with the type of viral vector used and are well-known in the art.

In one specific embodiment, synPCCB is be placed under the transcriptional control of a ubiquitous or tissue-specific promoter, with a 5′ intron, polyadenylation signal, and mRNA stability element, such as the woodchuck post-transcriptional regulatory element (WPRE) or hepatitis B post-transcriptional regulatory element (HPRE). The use of a tissue-specific promoter can restrict unwanted transgene expression, as well as facilitate persistent transgene expression. The therapeutic transgene is then delivered as coated or naked DNA into the systemic circulation, portal vein, or directly injected into a tissue or organ, such as the liver or kidney. In addition to the liver or kidney, the brain, pancreas, eye, heart, lungs, bone marrow, and muscle may constitute targets for therapy. Other tissues or organs may be additionally contemplated as targets for therapy.

In another embodiment, the same synPCCB expression construct is packaged into a viral vector, such as an adenoviral vector, retroviral vector, lentiviral vector, or adeno-associated viral vector, and delivered by various means into the systemic circulation, portal vein, or directly injected into a tissue or organ, such as the liver or kidney. In addition to the liver or kidney, the brain, pancreas, eye, heart, lungs, bone marrow, and muscle constitute targets for therapy. Other tissues or organs may be additionally contemplated as targets for therapy.

Tissue-specific promoters include, without limitation, might include Apo A-I, ApoE, hAAT, transthyretin, liver-enriched activator, albumin, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).

Regulable promoters (for example, ligand-inducible or stimulus-inducible promoters) are also contemplated for expression constructs according to the invention.

In yet another embodiment, synPCCB is used in ex vivo applications via packaging into a retro- or lentiviral vector to create an integrating vector that is used to permanently correct any cell type from a patient with PCC deficiency. The synPCCB-transduced and corrected cells is then used as a cellular therapy. Examples include CD34+ stem cells, primary hepatocytes, or fibroblasts derived from patients with PCC deficiency. Fibroblasts are reprogrammed to other cell types using iPS methods well known to practitioners of the art. In yet another embodiment, synPCCB is recombined using genomic engineering techniques that are well known to practitioners of the art, such as ZFNs and TALENS, into the PCCB locus, a genomic safe harbor site, such as AAVS1, or into another advantageous location, such as into rDNA, the albumin locus, GAPDH, or a suitable expressed pseudogene.

In one embodiment, the disclosure teaches a composition comprising the synthetic polynucleotide synPCCB in combination with PCCA and optionally synPCCAs to generate dual expression vectors. The disclosure teaches treatment providing for either type of PCC deficiency, A or B.

In one embodiment, the disclosure teaches creating an AAV that expresses both genes or a LNP that does likewise. In one embodiment, the disclosure teaches, but is not limited to, dual AAVs and/or dual LNP mRNA plus transposase mRNA plus CRISPR and GUIDE.

In yet another embodiment, the invention is directed at the production of viral vectors designed to express the PCCB polynucleotide(s). Examples of vectors include, but are not limited to, adenoassociated viral vectors (AAV), adenoviral vectors, retroviral vectors, lentiviral vectors, and herpes viral vectors. Examples of AAV serotypes include, but are not limited to AAV1, 2, 3, 3B, 4, 5, 6, 7, 8, 9, rh8, rh10, ANC80L65, LK03, NP59, and others well known to practitioners of the art.

Hybrid vectors that use a combination of AAV vectors and vectors, including non viral vectors such as lipid nanoparticles (LNPs), designed to deliver and express Piggy Bac (PB) transposons or other DNA transposons that use a cut and paste mechanism to accomplish insertion of the target sequence is yet another embodiment of the use of the polynucleotides described herein. Hybrid vectors include a AAV designed to express a viral transgene encoding a polynucleotides of described herein, either alone or in combination with a polynucleotide encoding the propionyl-CoA carboxylase alpha, and flanked by Piggy Bac (PB) transposon specific terminal repeats, when administered with a source of Piggy Bac (PB) transpose mRNA, will enable transposition of the viral transgene into the genome, with permanent correction of transduced cells resulting. Such cells include hepatocytes, the main cell of the liver, which is targeted by many AAV serotypes, AAV1, 2, 3, 3B, 4, 5, 6, 7, 8, 9, rh8, rh10, ANC80L65, LK03, NP59, and others well known to practitioners of the art.

In yet another embodiment, the invention is directed at the production of viral vectors designed to express the PCCB polynucleotide(s) and a polynucleotide encoding the propionyl-CoA carboxylase alpha. Examples of vectors include, but are not limited to, adenoassociated viral vectors (AAV), adenoviral vectors, retroviral vectors, lentiviral vectors, and herpes viral vectors. Examples of AAV serotypes include, but are not limited to AAV1, 2, 3, 3B, 4, 5, 6, 7, 8, 9, rh8, rh10, ANC80L65, LK03, NP59, and others well known to practitioners of the art.

In yet another embodiment, the invention is directed at the production of lipid nanoparticles designed to express the PCCB polynucleotide(s) and a polynucleotide encoding the propionyl-CoA carboxylase alpha.

In yet another embodiment, the invention is directed at the production of DNA plasmids designed to express the PCCB polynucleotide(s) and a polynucleotide encoding the propionyl-CoA carboxylase alpha. Such plasmids may be devoid of bacterial DNA and include forms of closed end DNA generated by AAV replication in a baculoviral system.

A composition (pharmaceutical composition) for treating an individual by gene therapy may comprise a therapeutically effective amount of a vector comprising the synPCCB transgenes or a viral particle produced by or obtained from same. The pharmaceutical composition may be for human or animal usage. Typically, a physician will determine the actual dosage which will be most suitable for an individual subject, and it will vary with the age, weight, and response of the particular individual.

The composition may, in specific embodiments, comprise a pharmaceutically acceptable carrier, diluent, excipient, or adjuvant. Such materials should be non-toxic and should not interfere with the efficacy of the transgene. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences [Mack Pub. Co., 18th Edition, Easton, Pa. (1990)]. The choice of pharmaceutical carrier, excipient, or diluent can be selected with regard to the intended route of administration and standard pharmaceutical practice. The pharmaceutical compositions may comprise as, or in addition to, the carrier, excipient, or diluent any suitable binder(s), lubricant(s), suspending agent(s), coating agent(s), solubilizing agent(s), and other carrier agents that may aid or increase the viral entry into the target site (such as for example a lipid delivery system). For oral administration, excipients such as starch or lactose may be used. Flavoring or coloring agents may be included, as well. For parenteral administration, a sterile aqueous solution may be used, optionally containing other substances, such as salts or monosaccharides to make the solution isotonic with blood.

A composition according to the invention may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, modulators, or drugs (e.g., antibiotics).

The composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. Additional dosage forms contemplated include: in the form of a suppository or pessary; in the form of a lotion, solution, cream, ointment or dusting powder; by use of a skin patch; in capsules or ovules; in the form of elixirs, solutions, or suspensions; in the form of tablets or lozenges.

### Examples

Cell culture studies: Five synthetic codon-optimized human propionyl-CoA carboxylase subunit beta genes (synPCCB1-5) were engineered using an iterative approach, wherein the naturally occurring PCCB cDNA (NCBI Reference Sequence: NM_000532.4) was optimized codon by codon to create (synPCCB1-5) (SEQ ID NOs:2-6) a variety of codon optimization methods, one of which incorporated critical factors involved in protein expression, such as codon adaptability, mRNA structure, and various cis-elements in transcription and translation. The resulting sequences were manually inspected and subject to expert adjustment. The synPCCB alleles displayed maximal divergence from the PCCB cDNA at the nucleotide level yet retained optimally utilized codons at each position.

To improve the expression of propionyl-CoA carboxylase and create a vector that express the human PCCB gene in a more efficient fashion, synPCCB1 and synPCCB2 were cloned into an AAV vector backbone containing the strong elongation factor 1 alpha (EF1a) promoter, and transfected into 293 cells, which are human transformed kidney cells. Plasmid DNA was transfected into 293 cells with Lipofectamine 2000™ (ThermoFisher™). Protein was extracted in T-Per buffer (Peirce™). Western blots were performed with 20 micrograms of total protein and stained with PCCB antibody (Abcam™ ab186836) at a dilution of 1 to 2,000 and beta-actin (Abcam™ ab227387) at a dilution of 1 to 2,000. The secondary antibody (GE Healthcare™, NA934) was stained at a concentration of 1 to 30,000.

Cloning and transfection methods are well understood by practitioners of the art (Sambrook, Fritsch, Maniatis. Molecular Cloning: A Laboratory Manual). After 48 hours, cellular protein was extracted from the transfected cells and evaluated for propionyl-CoA carboxylase protein expression using Western analysis (Chandler, et al. 2010 Mol Ther 18:11-6). The results show that synPCCB1 is transcribed and translated as or more efficiently than PCCB (FIG. 2).

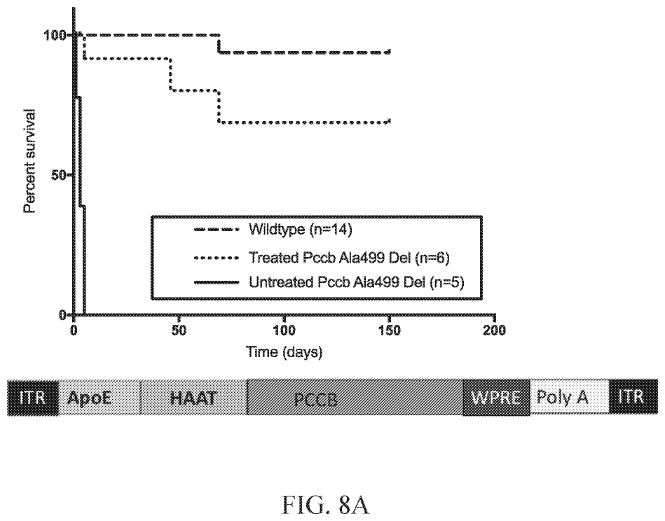

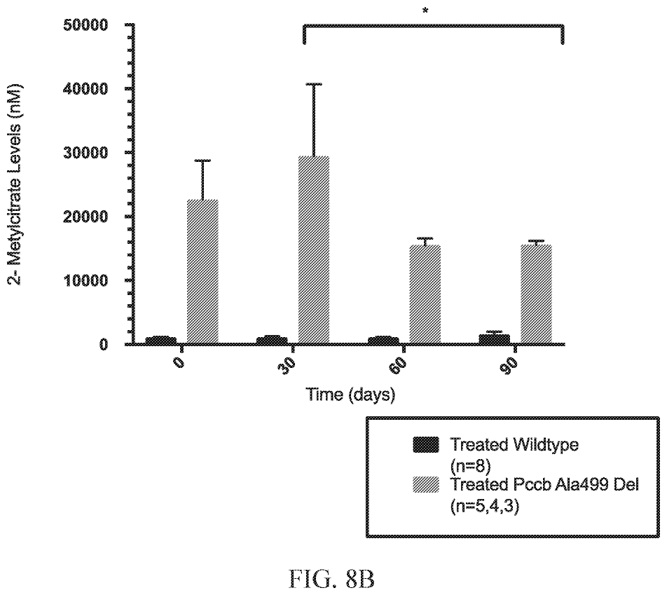

Gene therapy in propionyl-CoA carboxylase Knock-out (Pccb^{-/-}) Mice. The promising expression data from both constructs led to the production of AAV9-EF1A-synPCCB1 (SEQ ID: 9) and AAV9-hAAT synPCCB1 WPRE (SEQ ID: 11) which were delivered to neonatal Pccb^{-/-} mice. As presented in FIG. 7A, 50% of the Pccb^{-/-} mice that received AAV9-EF1A-synPCCB1 lived to 30 days, and further had an improved clinical appearance, as compared to the untreated Pccb^{-/-} mice which had 100% mortality in early life. A substantial reduction in the disease related metabolite methylcitrate accompanied the rescue as seen in FIG. 7B. Similar results were observed after treatment with AAV9-hAAT synPCCB1 WPRE. It should be noted that the antibody used for Western blotting can detect both human (PCCB) and murine (Pccb) enzymes.

Animal studies were reviewed and approved by the National Human Genome Research Institute Animal User Committee. Hepatic injections were performed on non-anesthetized neonatal mice, typically within several hours after birth. Viral particles were diluted to a total volume of 20 microliters with phosphate-buffered saline immediately before injection and were delivered into the liver parenchyma using a 32-gauge needle and transdermal approach, as previously described.

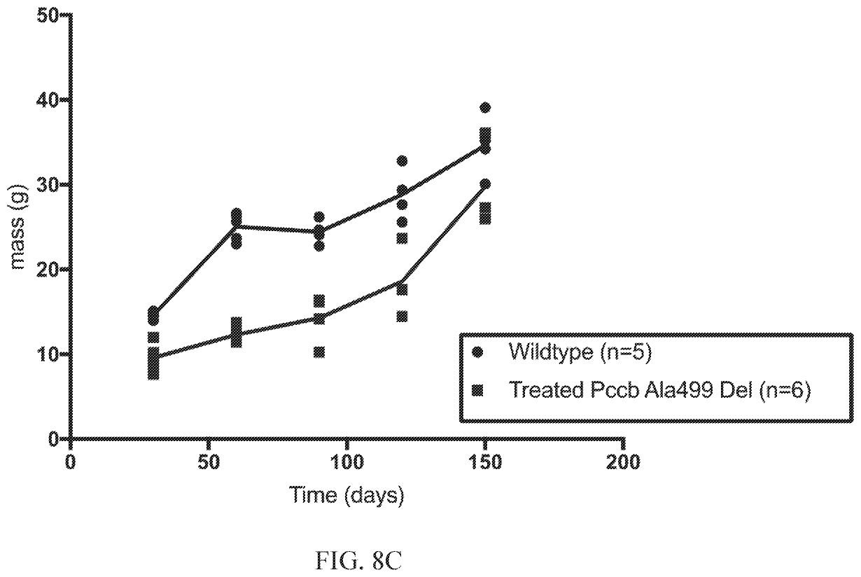

Treatment with synPCCB polynucleotide delivered using an AAV (adeno-associated virus) rescued the Pccb^{-/-} mice from neonatal lethality (FIG. 7A, 8A), improved their growth (FIG. 8 C), and lowered the levels of plasma methylcitrate in the blood (FIG. 7B, 8B). This establishes the pre-clinical efficacy of synPCCB as a treatment for PA in vivo, including in other animal models, as well as in humans.

As a body of data, the aggregate results show that representative members of the synPCCB family express PCCB at an increased level in human cells compared to the wild type PCCB gene. In a further stringent in vivo test, synPCCB1 was configured into potent gene therapy vectors that effectively treated a lethal mouse model of PA. These vectors used promoters that resulted in ubiquitous or liver-restricted gene expression, and represent two classes of new gene therapy treatments for propionic acidemia caused by PCCB mutations. Gene therapy vectors and genome editing cassettes for human translation using these synPCCB alleles is indicated.

1. A synthetic propionyl-CoA carboxylase subunit beta (PCCB) polynucleotide (synPCCB) selected from the group consisting of:
a) a polynucleotide comprising the nucleic acid sequence of any one of SEQ ID NOs: 2-6; and
b) a polynucleotide having a nucleic acid sequence with at least about 80% identity to the nucleic acid sequence of any one of SEQ ID NOs: 2-6 and encoding a polypeptide according to SEQ ID NO:7, and having equivalent or increased expression in a host as compared to either expression of any one of SEQ ID NOs: 2-6 or SEQ ID NO:1 expression, wherein the polynucleotide does not have the nucleic acid sequence of SEQ ID NO:1.

2. The synthetic polynucleotide of claim 1, wherein:
(a) the polynucleotide has at least about 90% identity to the nucleic acid sequence of any one of SEQ ID NOs: 2-6;
(b) the polynucleotide has at least about 95% identity to the nucleic acid sequence of any one of SEQ ID NOs: 2-6;
(c) the sequence selected from the group consisting of SEQ ID NOs: 2-6 exhibits increased expression in an appropriate host relative to the expression of SEQ ID NO:1 in an appropriate host;
(d) the polynucleotide comprises a synthetic PCCB gene flanked by a 5′ untranslated region (5′UTR) that includes a strong Kozak translational initiation signal;
(e) the polynucleotide further comprises a wood chuck post-translational response element (SEQ ID: 30) or a hepatitis post-translational response element (SEQ ID:31);
(f) the synthetic polynucleotide further comprises a polyadenylation signal;
(g) the synthetic polynucleotide further comprises a donor cassette that targets the stop codon of human albumin, which yields, after homologous recombination synPCCB1 fused via a P2 peptide to the carboxy terminus of albumin;
(h) the synthetic polynucleotide further comprises an integrating AAV vector that uses homologous recombination to insert synPCCB1 into human albumin (SEQ ID NO: 36); or
(i) the polynucleotide comprises a synthetic PCCB gene configured to integrate into a genome after delivery using a lentiviral vector.

3-4. (canceled)

5. The synthetic polynucleotide of claim 1, wherein the synthetic polynucleotide having increased expression comprises a nucleic acid sequence comprising codons that have been optimized relative to the naturally occurring human propionyl-CoA carboxylase subunit beta polynucleotide sequence (SEQ ID NO:1).

6. The synthetic polynucleotide of claim 1, wherein the nucleic acid sequence has at least about 70% of less commonly used codons replaced with more commonly used codons.

7. A recombinant expression vector comprising the synthetic polynucleotide of claim 1.

8. The recombinant vector of claim 7, wherein the vector is a recombinant adeno-associated virus (rAAV), said rAAV comprising an AAV capsid, and a vector genome packaged therein, said vector genome comprising:
(a) a 5′-inverted terminal repeat sequence (5′-ITR) sequence;
(b) a promoter sequence;
(c) a partial fragment or complete coding sequence for PCCB; and
(d) a 3′-inverted terminal repeat sequence (3′-ITR) sequence.

9. The rAAV according to claim 8, wherein:
(a) the vector is comprised of the structure in FIG. 1;
(b) the AAV capsid is from an AAV of a serotype selected from the group consisting of AAV1, 2, 3, 3B, 4, 5, 6, 7, 8, 9, 44.9, rh8, rh10, ANC80L65, LK03, NP59, KP1, and mutants thereof;
(c) wherein the rAAV further comprises terminal repeat sequences recognized by piggyBac transposase;
(d) wherein the promotor is a tissue specific promoter;
(e) the rAAV further comprises terminal repeat sequences (SEQ ID: 32-33) from the piggyBac transposon, located after the 5′AAV ITR and before the 3′ AAV ITR,
(f) the expression vector is AAV2/9-CBA-synPCCB1;
(g) the expression vector is AAV2/9-EF1L-synPCCB1;
(h) the expression vector is AAV2/9-EF1S-HPRE synPCCB1; or
(i) the expression vector is AAV2/9-hAAT-WPRE synPCCB1 or AAV2/9-HCR-hAAT-synPCCB1 RBG.

10-13. (canceled)

14. The rAAV according to claim 8, wherein the promoter is selected from the group consisting of chicken-beta actin promoter (SEQ ID NO: 8), elongation factor 1 alpha long promoter (EF1AL) (SEQ ID NO:9), elongation factor 1 alpha short promoter with a 3′ hepatitis B post translation response element (HPRE) (SEQ ID NO:10), human alpha 1 antitrypsin promoter with a 3′ woodchuck post translation response element (WPRE) (SEQ ID NO:11), long (SEQ ID NO:13) and short variants (SEQ ID NO:12) of apolipoprotein E enhancer, long (SEQ ID NO:15) and short variants of human alpha 1 antitrypsin promoter (SEQ ID NO:14), enhanced human alpha 1 antitrypsin promoter (SEQ ID:35), and enhanced TBG promoter (SEQ ID:34),
wherein the polynucleotide optionally comprises at least one intron selected from the group consisting of a chimeric intron (SEQ ID NO:16), modified B-globin intron (SEQ ID NO: 17), and a synthetic intron (SEQ ID NO:18).

15-16. (canceled)

17. The rAAV according to claim 8, wherein:
(a) the promoter is an apolipoprotein E enhancer and a human alpha 1 antitrypsin promoter operably linked to form a short (SEQ ID NO: 19) or long liver specific enhancer-promoter unit (SEQ ID NO: 20) and placed 5′ to an intron selected from SEQ ID NO:16-18;
(b) the promoter is a liver specific enhancer derived from sequences upstream of the alpha-1-microglobulin/bikunin precursor (SEQ ID:22 and SEQ ID:23) and operably linked to the human thyroxine-binding globulin promoter (TBG) (SEQ ID:24); or
(c) the promoter is a liver specific enhancer and human thyroxine-binding globulin promoter of SEQ ID:25;
optionally wherein:
(i) the intron is the modified β-globin intron (SEQ ID NO: 17); or
(ii) the intron comprises a polynucleotide having the sequence of SEQ ID NO:21.

18-22. (canceled)

23. The synthetic polynucleotide of claim wherein:
(a) the synthetic polynucleotide further comprising an internal ribosome entry site (IRES) (SEQ ID: 26) instead of, or in addition to, a UTR; or
(b) the UTR comprises sequences selected from the group consisting of human albumin (SEQ ID: 27), SERPINA 1 (SEQ ID: 28), and SERPINA 3 (SEQ ID: 29);
optionally wherein the synthetic polynucleotide further comprises at least one translation enhancer element (TEE).

24. (canceled)

25. The synthetic polynucleotide of claim 23, wherein:
(a) the TEE is located between the promoter and the start codon; or
(b) the 5′UTR comprises a TEE.

26-29. (canceled)

30. The synthetic polynucleotide of claim 1, wherein the polyadenylation signal is a rabbit beta globin gene or the bovine growth hormone gene.

31-33. (canceled)

34. The synthetic polynucleotide of claim 1, further comprising an integrating AAV vector that uses homologous recombination to insert synPCCB1 into the 5′ end of human albumin (SEQ ID NO: 37).

35. (canceled)

36. The synthetic polynucleotide of claim 1, wherein lentiviral vector further comprises:
(a) a human alpha 1 antitrypsin enhancer having the sequence SEQ ID:38; or
(b) the elongation factor 1 long promoter having the sequence SEQ ID:39.

37-38. (canceled)

39. The vector of claim 9, wherein the tissue specific promoter is selected from the group consisting of Apo A-I, ApoE, hAAT, transthyretin, liver-enriched activator, albumin, TBG, PEPCK, and RNAP_{II} promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).

40-43. (canceled)

44. A composition comprising the synthetic polynucleotide of claim 1 or a recombinant expression vector comprising the polynucleotide and a pharmaceutically acceptable carrier, optionally wherein the composition further comprises a hybrid AAV-piggyBac transposon system.

45-46. (canceled)

47. A method of treating a disease or condition mediated by propionyl-CoA carboxylase, comprising administering to a subject in need thereof a therapeutic amount of the synthetic polynucleotide of claim 1.

48. A method of treating a disease or condition mediated by propionyl-CoA carboxylase, comprising administering to a subject a propionyl-CoA carboxylase produced using the synthetic polynucleotide of claim 1 or a recombinant expression vector comprising the synthetic polynucleotide.

49. The method of claim 47, wherein:
(a) the disease or condition is propionic acidemia (PA);
(b) the polynucleotide is inserted into the cell of the subject via genome editing on the cell of the subject using a nuclease selected from the group of zinc finger nucleases (ZFNs), transcription activator-like effector nucleases (TALENs), the clustered regularly interspaced short palindromic repeats (CRISPER/cas system) and meganuclease re-engineered homing endonucleases on a cell from the subject; and the method further comprises administering the cell to the subject; or
(c) the composition is administered through the route consisting of subcutaneously, intramuscularly, intradermally, intraperitoneally, and intravenously;

50-53. (canceled)

54. The method of claim 48, wherein the recombinant expression vector is a recombinant adeno-associated virus (rAAV), and wherein:
(a) the rAAV is administered at a dose of about 1×10¹¹ to about 1×10¹⁴ genome copies (GC)/kg;
(b) administering the rAAV comprises administration of a single dose of rAAV; or
(c) administering the rAAV comprises administration of multiple doses of rAAV.

55-56. (canceled)


SEQUENCE LISTING (40 sequences)

SEQ ID NO: 1 - DNA, 2187 bp, Homo sapiens
atggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatga

SEQ ID NO: 2 - DNA, 2112 bp, Artificial Sequence
  Synthetic construct
atgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtga

SEQ ID NO: 3 - DNA, 2187 bp, Artificial Sequence
  Synthetic construct
atggccggcttctgggtggggactgctccccttgtcgccgcaggacgcagaggccgctggcctcctcagcagctcatgctctcagcagctctgaggaccctgaaacacgtgctttactacagtcgacagtgtctgatggtgtctaggaacctgggtagcgtgggctatgatcccaatgaaaagacctttgacaaaatactggtcgctaatagaggggaaattgcttgtcgcgtgatacggacgtgcaagaagatgggtatcaaaaccgtggcaattcactctgacgttgatgcttcctcagtgcatgtaaagatggcggatgaggctgtttgcgtgggtccagcacctacaagcaagagctatctcaacatggatgccatcatggaagctatcaagaaaacccgtgcacaagctgtgcatccagggtatggctttctctccgagaacaaagaatttgcccggtgtctggcagcggaggacgtggtgttcattgggcctgatacgcatgcaattcaagccatgggcgataagattgagagcaagctgcttgctaagaaagcagaagttaacacaatcccaggctttgacggcgttgtcaaagacgccgaagaagcggtacgtattgcccgagaaatcggctaccccgttatgatcaaggcgtcagccggaggtggaggaaaagggatgaggattgcctgggatgacgaggagactagggatgggttccggctctccagtcaggaagcagcatcttcttttggtgacgatagactgctgatagagaaattcatcgacaaccctcgacacattgaaatccaggtactgggagacaaacacggaaatgcactttggctcaatgaacgcgagtgctccattcagcgcaggaaccagaaagtggtcgaggaagcaccctccatcttcctggatgccgagacaaggcgcgctatgggcgagcaggccgttgcactcgctagagccgtgaagtactcttctgcgggtaccgtggaatttctggtagacagcaagaagaacttctatttcctggagatgaatacccggctgcaagtcgagcatccagtcactgagtgtataactggcctggacctggtacaggaaatgattcgtgtagcgaagggatacccgctccggcacaaacaagccgacattcgcatcaatgggtgggctgtggagtgcagagtctatgcagaggatccctataagtccttcgggcttccctccataggcaggcttagtcagtatcaggagccattgcacttgcctggcgtcagggtggactccggcatccaaccgggcagcgacatttcaatttactacgatcccatgatcagcaagttgattacctatggatctgaccggacagaggctctgaagagaatggccgacgccctggacaattacgtgataagaggagtgacacacaacattgccctgttgcgggaggtgatcatcaatagcagattcgtgaagggtgacatctccaccaagttcctgagtgacgtataccccgacggctttaaggggcatatgctgacaaagtcagagaagaatcaactcctcgcaatagccagtagcctgtttgttgccttccagctgagggctcagcacttccaggagaatagcagaatgcccgttatcaaacctgatatcgcgaattgggaattgagcgtgaagctgcacgataaagttcatactgttgtggcctcaaacaatggaagcgtctttagcgtggaggtcgatggatccaaactgaacgtgaccagtacctggaatttggccagtccgctgttgtctgtctccgtggatggaacgcaacgaactgtgcagtgtctgtctcgcgaagccggaggcaacatgagcattcagtttctcgggactgtgtacaaagtcaacatcctgacccgactggctgccgagctgaacaaatttatgcttgagaaagtcactgaggatacgtctagcgtccttcggagtcctatgccaggggtggtggtggccgtttcagtcaaaccaggtgatgccgtagccgaaggtcaggaaatctgcgttatcgaggctatgaagatgcagaacagcatgacagccgggaaaaccggaacagtgaagtcagttcattgccaggctggggacacagtcggcgagggcgatttgctggtggaactggaatga

SEQ ID NO: 4 - DNA, 2187 bp, Artificial Sequence
  Synthetic construct
atggccggattttgggttggaacagctcctctggtggccgctgggagaagaggaagatggcctcctcagcagctgatgctgtctgccgctctgagaaccctgaaacacgtgctgtactacagccggcagtgcctgatggtgtccagaaatctgggcagcgtgggctacgaccccaacgagaaaaccttcgacaagatcctggtggccaaccggggagagatcgcctgcagagtgatccggacctgcaagaagatgggcatcaagaccgtggccatccactccgatgtggatgcctctagcgtgcacgtgaaaatggccgatgaggccgtgtgtgtgggccctgctcctacaagcaagagctacctgaacatggacgccatcatggaagccattaagaaaacaagagcccaggccgtgcatcccggctacggatttctgagcgagaacaaagaatttgcccggtgcctggccgccgaggacgtggtgtttattggccctgatacacacgccatccaggccatgggcgataagatcgagtctaagctgctggccaagaaagccgaagtgaacacaatccccggcttcgacggcgtggtcaaggatgctgaagaagccgtgcggatcgccagagaaatcggctaccccgtgatgatcaaagcctctgctggcggaggcggcaagggaatgagaatcgcctgggacgacgaagagacacgcgacggctttagactgagcagccaagaagccgccagctccttcggagatgacagactgctgatcgagaagttcatcgacaaccccagacacatcgagatccaggtgctgggcgacaagcacggaaatgccctgtggctgaacgagagagagtgcagcatccagcggcggaaccagaaagtggtggaagaggcccctagcatcttcctggacgccgaaactcggagagccatgggagaacaggctgtggctctggctagagccgtgaagtatagcagcgccggcaccgtggaatttctggtggacagcaagaagaacttctacttcctcgagatgaacacccggctgcaggtcgagcaccctgtgaccgagtgtatcacaggcctggacctggtgcaagagatgatcagagtggccaagggctaccctctgagacacaagcaggccgacatccggatcaatggctgggccgttgagtgcagagtgtacgccgaggatccctacaagagcttcggcctgcctagcatcggccggctgtctcagtatcaagagccactgcatctgcccggcgtcagagtggattctggaatccagcctggcagcgacatcagcatctactacgaccctatgatctccaagctgatcacctacggcagcgaccggacagaggccctgaagagaatggctgacgccctggacaactacgtgatcagaggcgtgacccacaatatcgccctgctgcgggaagtgatcatcaacagcagattcgtgaaaggcgatatcagcaccaagtttctgtccgacgtgtaccccgacggcttcaagggacacatgctgaccaagagcgagaagaaccagctgctcgccattgcctccagcctgtttgtggcctttcagctgagagcccagcacttccaagagaacagcagaatgcccgtgatcaagcccgatatcgccaactgggagctgagcgtgaagctgcacgataaggtgcacacagtggtggccagcaacaacggctccgtgttcagcgtggaagtggacggcagcaagctgaacgtgacctccacctggaatctggcctctccactgctgtccgtgtctgtggatggcacccagagaaccgtgcagtgtctgagcagagaagcaggcggcaatatgagcatccagtttctgggcaccgtgtacaaagtgaacatcctgaccagactggccgctgagctgaacaagttcatgctggaaaaagtgaccgaggacaccagcagcgtgctgagatctcctatgcctggtgtcgtggtggccgtgtcagtgaaacctggggatgctgtggccgagggccaagagatctgtgtgatcgaggccatgaagatgcagaacagcatgaccgccggcaagaccggcacagtgaagtctgtgcattgtcaggccggcgatacagtcggagaaggcgatctgctggtggaactggaatga

SEQ ID NO: 5 - DNA, 2187 bp, Artificial Sequence
  Synthetic construct
atggccggcttctgggtgggcaccgcccccctggtggccgccggcagaagaggcagatggcccccccagcagctgatgctgagcgccgccctgagaaccctgaagcacgtgctgtactacagcagacagtgcctgatggtgagcagaaacctgggcagcgtgggctacgaccccaacgagaagaccttcgacaagatcctggtggccaacagaggcgagatcgcctgcagagtgatcagaacctgcaagaagatgggcatcaagaccgtggccatccacagcgacgtggacgccagcagcgtgcacgtgaagatggccgacgaggccgtgtgcgtgggccccgcccccaccagcaagagctacctgaacatggacgccatcatggaggccatcaagaagaccagagcccaggccgtgcaccccggctacggcttcctgagcgagaacaaggagttcgccagatgcctggccgccgaggacgtggtgttcatcggccccgacacccacgccatccaggccatgggcgacaagatcgagagcaagctgctggccaagaaggccgaggtgaacaccatccccggcttcgacggcgtggtgaaggacgccgaggaggccgtgagaatcgccagagagatcggctaccccgtgatgatcaaggccagcgccggcggcggcggcaagggcatgagaatcgcctgggacgacgaggagaccagagacggcttcagactgagcagccaggaggccgccagcagcttcggcgacgacagactgctgatcgagaagttcatcgacaaccccagacacatcgagatccaggtgctgggcgacaagcacggcaacgccctgtggctgaacgagagagagtgcagcatccagagaagaaaccagaaggtggtggaggaggcccccagcatcttcctggacgccgagaccagaagagccatgggcgagcaggccgtggccctggccagagccgtgaagtacagcagcgccggcaccgtggagttcctggtggacagcaagaagaacttctacttcctggagatgaacaccagactgcaggtggagcaccccgtgaccgagtgcatcaccggcctggacctggtgcaggagatgatcagagtggccaagggctaccccctgagacacaagcaggccgacatcagaatcaacggctgggccgtggagtgcagagtgtacgccgaggacccctacaagagcttcggcctgcccagcatcggcagactgagccagtaccaggagcccctgcacctgcccggcgtgagagtggacagcggcatccagcccggcagcgacatcagcatctactacgaccccatgatcagcaagctgatcacctacggcagcgacagaaccgaggccctgaagagaatggccgacgccctggacaactacgtgatcagaggcgtgacccacaacatcgccctgctgagagaggtgatcatcaacagcagattcgtgaagggcgacatcagcaccaagttcctgagcgacgtgtaccccgacggcttcaagggccacatgctgaccaagagcgagaagaaccagctgctggccatcgccagcagcctgttcgtggccttccagctgagagcccagcacttccaggagaacagcagaatgcccgtgatcaagcccgacatcgccaactgggagctgagcgtgaagctgcacgacaaggtgcacaccgtggtggccagcaacaacggcagcgtgttcagcgtggaggtggacggcagcaagctgaacgtgaccagcacctggaacctggccagccccctgctgagcgtgagcgtggacggcacccagagaaccgtgcagtgcctgagcagagaggccggcggcaacatgagcatccagttcctgggcaccgtgtacaaggtgaacatcctgaccagactggccgccgagctgaacaagttcatgctggagaaggtgaccgaggacaccagcagcgtgctgagaagccccatgcccggcgtggtggtggccgtgagcgtgaagcccggcgacgccgtggccgagggccaggagatctgcgtgatcgaggccatgaagatgcagaacagcatgaccgccggcaagaccggcaccgtgaagagcgtgcactgccaggccggcgacaccgtgggcgagggcgacctgctggtggagctggagtga

SEQ ID NO: 6 - DNA, 2187 bp, Artificial Sequence; identical to SEQ ID NO: 5
  Synthetic construct

SEQ ID NO: 7 - protein, 539 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 8 - DNA, 6795 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 9 - DNA, 6688 bp, Artificial Sequence
  synthetic
ccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtagccatgctctagcgatcgcggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcgccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagagacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatcgaattccctgcaggtagagcatggctacgtaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 10 - DNA, 7161 bp, Artificial Sequence
  synthetic
gggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagcggcaccgcacctctggtcgccgcaggacgcaggggaagatggcctccacagcagctgatgctgagcgcagccctgaggaccctgaagcacgtgctgtactattctaggcagtgcctgatggtcagccgcaacctgggcagcgtgggatacgaccctaatgagaagacattcgataaaatcctggtggctaaccgcggcgaaatcgcatgccgagtgattcggacctgtaagaaaatggggatcaagacagtcgccattcacagcgacgtggatgccagcagcgtccatgtgaagatggcagacgaggccgtctgcgtgggaccagcccctacatctaaaagttacctgaacatggatgctatcatggaagcaattaagaaaactagggcccaggctgtgcaccctggctatgggttcctgagcgagaataaggaatttgcacgatgtctggcagctgaggacgtggtctttatcggaccagatacacatgctattcaggcaatgggcgacaagatcgagtccaaactgctggccaagaaagctgaagtgaatactatccccgggttcgacggagtggtcaaggatgcagaggaagccgtgagaatcgccagggagattggctaccctgtgatgattaaggcatctgccggcgggggaggcaaagggatgaggatcgcctgggacgatgaggaaactcgcgatggatttcgactgtctagtcaggaagcagccagcagcttcggcgacgataggctgctgatcgagaagttcattgacaacccccgccacatcgaaattcaggtgctgggggataaacatggaaacgccctgtggctgaatgagcgggaatgtagcattcagcggagaaatcagaaggtggtcgaggaagctccttccatctttctggacgccgagacaaggcgcgctatgggagaacaggctgtcgcactggccagagctgtgaaatactcctctgccggcactgtcgagttcctggtggacagcaagaaaaacttctattttctggaaatgaacacccggctgcaggtcgagcacccagtgactgaatgcattaccgggctggatctggtccaggagatgatcagagtggccaagggataccccctgcgacataaacaggctgacatccggattaacggctgggcagtcgagtgtcgggtgtacgccgaagatccatataagtctttcggactgcccagtattggccgactgtcacagtatcaggagcctctgcacctgccaggcgtcagagtggacagcggcatccagcctgggtccgacatctctatctactatgatccaatgatcagcaagctgattacatacggctccgatcggactgaggccctgaaaagaatggcagacgccctggataactatgtcattagaggggtgacccataatatcgctctgctgagagaagtcatcattaactccaggttcgtgaagggagacatcagcaccaaatttctgtccgacgtgtaccccgatggcttcaaggggcacatgctgacaaagtctgagaaaaatcagctgctggctatcgcaagttcactgttcgtggcatttcagctgcgggcccagcattttcaggagaacagtagaatgcccgtgatcaagcctgacattgcaaattgggaactgagtgtcaagctgcacgataaagtgcataccgtggtcgcttcaaacaatggcagcgtgttcagcgtcgaggtggacgggtctaaactgaacgtgaccagtacatggaatctggcctcaccactgctgtcagtcagcgtggatggcacacagcgcactgtgcagtgcctgagccgggaggcaggaggaaacatgagtattcagtttctggggactgtctataaggtgaacatcctgaccaggctggctgcagaactgaataagttcatgctggagaaagtgaccgaagacacaagctccgtgctgcgctcaccaatgccaggagtggtcgtggccgtcagcgtgaagccaggggatgcagtggctgagggacaggagatttgcgtgattgaggctatgaaaatgcagaacagcatgaccgcaggaaagactggcaccgtgaaaagcgtgcattgtcaggctggggatactgtcggggaaggggatctgctggtggaactggagtgaagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 11 - DNA, 6193 bp, Artificial Sequence
  synthetic
  misc_feature (3022)..(3022): n is a, c, g, or t
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagtcctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgcccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgacgcgaattcgcccttcaccatggcggcggcattacgggtggcggcggtcggggcaaggctcagcgttctggcgagcggtctccgcgccgcggtccgcagcctttgcagccaggccacctctgttaacgaacgcatcgaaaacaagcgccggaccgcgctgctgggagggggccaacgccgtattgacgcgcagcacaagcgaggaaagctaacagccagggagaggatcagtctcttgctggaccctggcagctttgttgagagcgacatgtttgtggaacacagatgtgcagattttggaatggctgctgataagaataagtttcctggagacagcgtggtcactggacgaggccgaatcaatggaagattggtttatgtcttcagtcaggattttacagtttttggaggcagtctgtcaggagcacatgcccaaaagatctgcaaaatcatggaccaggccataacggtgggggctccagtgattgggctgaatgactctgggggagcacggatccaagaaggagtggagtctttggctggctatgcagacatctttctgaggaatgttacggcatccggagtcatccctcagatttctctgatcatgggcccatgtgctggtggggccgtctactccccagccctaacagacttcacgttcatggtaaaggacacctcctacctgttcatcactggccctgatgttgtgaagtctgtcaccaatgaggatgttacccaggaggagctcggtggtgccaagacccacaccaccatgtcaggtgtggcccacagagcttttgaaaatgatgttgatgccttgtgtaatctccgggatttcttcaactacctgcccctgagcagtcaggacccggctcccgtccgtgagtgccacgatcccagtgaccgtctggttcctgagcttgacacaattgtccctttggaatcaaccaaagcctacaacatggtggacatcatacactctgttgttgatgagcgtgaattttttgagatcatgcccaattatgccaagaacatcattgttggttttgcaagaatgaatgggaggactgttggaattgttggcaaccaacctaaggtggcctcaggatgcttggatattaattcatctgtgaaaggggctcgttttgtcagattctgtgatgcattcaatattccactcatcacttttgttgatgtccctggctttctacctggcacagcacaggaatacgggggcatcatccggcatggtgccaagcttctctacgcatttgctgaggcaactgtacccaaagtcacagtcatcaccaggaaggcctatggaggtgcctatgatgtcatgagctctaagcacctttgtggtgataccaactatgcctggcccaccgcagagattgcagtcatgggagcaaagggcgctgtggagatcatcttcaaagggcatgagaatgtggaagctgctcaggcagagtacatcgagaagtttgccaaccctttccctgcagcagtgcgagggtttgtggatgacatcatccaaccttcttccacacgtgcccgaatctgctgtgacctggatgtcttggccagcaagaaggtacaacgtccttggagaaaacatgcaaatattccattgtaaaagggcgaattccagcacactggcggccgttactagtggatccgagctcggtaccaagcttatcgataatcaacctctggattacaaaatttgtgaaagattgactggtattcttaactatgttgctccttttacgctatgtggatacgctgctttaatgcctttgtatcatgctattgcttcccgtatggctttcatttttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggcctcgaggacggggtgaactacgcctgangatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 12 - DNA, 189 bp, Artificial Sequence
  synthetic
cctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagag

SEQ ID NO: 13 - DNA, 318 bp, Artificial Sequence
  synthetic
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgaga

SEQ ID NO: 14 - DNA, 251 bp, Artificial Sequence
  synthetic
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtg

SEQ ID NO: 15 - DNA, 392 bp, Artificial Sequence
  synthetic
tgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 16 - DNA, 133 bp, Artificial Sequence
  synthetic
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 17 - DNA, 447 bp, Artificial Sequence
  synthetic
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 18 - DNA, 165 bp, Artificial Sequence
  synthetic
gtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcag

SEQ ID NO: 19 - DNA, 189 bp, Artificial Sequence; identical to SEQ ID NO: 12
  synthetic

SEQ ID NO: 20 - DNA, 710 bp, Artificial Sequence
  synthetic
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 21 - DNA, 1157 bp, Artificial Sequence
  synthetic
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaaggaattc

SEQ ID NO: 22 - DNA, 100 bp, Artificial Sequence
  synthetic
aggttaatttttaaaaagcagtcaaaagtccaagtggcccttggcagcatttactctctctgtttgctctggttaataatctcaggagcacaaacattcc

SEQ ID NO: 23 - DNA, 206 bp, Artificial Sequence
  synthetic
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattcc

SEQ ID NO: 24 - DNA, 460 bp, Artificial Sequence
  synthetic
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 25 - DNA, 666 bp, Artificial Sequence
  synthetic
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccgggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 26 - DNA, 588 bp, Artificial Sequence
  synthetic
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 27 - DNA, 63 bp, Artificial Sequence
  synthetic
gggagtatattagtgctaatttccctccgtttgtcctagcttttctcttctgtcaaccccaca

SEQ ID NO: 28 - DNA, 81 bp, Artificial Sequence
  synthetic
gggattcatgaaaatccactactccagacagacggctttggaatccaccagctacatccagctccctgagcagagttgaga

SEQ ID NO: 29 - DNA, 264 bp, Artificial Sequence
  synthetic
gggacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgaca

SEQ ID NO: 30 - DNA, 598 bp, Artificial Sequence
  synthetic
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 31 - DNA, 726 bp, Artificial Sequence
  synthetic
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 32 - DNA, 313 bp, Artificial Sequence
  synthetic
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 33 - DNA, 235 bp, Artificial Sequence
  synthetic
tttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 34 - DNA, 5832 bp, Artificial Sequence
  synthetic
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcagccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 35 - DNA, 5272 bp, Artificial Sequence
  synthetic
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcgcgccaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgtcgagaattcagccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 36 - DNA, 4093 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagctagtaacggccgccagtgtgctggaattcggcttggtcttcccaccaactccatgaaagtggattttattatcctcatcatgcagatgagaatattgagacttatagcggtatgcctgagccccaaagtactcagagttgcctggctccaagatttataatcttaaatgatgggactaccatccttactctctccatttttctatacgtgagtaatgttttttctgttttttttttttctttttccattcaaactcagtgcacttgttgagcttgtgaaacacaagcccaaggcaacaaaagagcaactgaaagctgttatggatgatttcgcagcttttgtagagaagtgctgcaaggctgacgataaggagacctgctttgccgaggaggtactacagttctcttcattttaatatgtccagtattcatttttgcatgtttggttaggctagggcttagggatttatatatcaaaggaggctttgtacatgtgggacagggatcttattttacaaacaattgtcttacaaaatgaataaaacagcactttgtttttatctcctgctctattgtgccatactgttaaatgtttataatgcctgttctgtttccaaatttgtgatgcttatgaatattaataggaatatttgtaaggcctgaaatattttgatcatgaaatcaaaacattaatttatttaaacatttacttgaaatgtggtggtttgtgatttagttgattttataggctagtgggagaatttacattcaaatgtctaaatcacttaaaattgccctttatggcctgacagtaacttttttttattcatttggggacaactatgtccgtgagcttccgtccagagattatagtagtaaattgtaattaaaggatatgatgcacgtgaaatcactttgcaatcatcaatagcttcataaatgttaattttgtatcctaatagtaatgctaatattttcctaacatctgtcatgtctttgtgttcagggtaaaaaacttgttgctgcaagtcaagctgccttaggcttaggaagcggcgccaccaatttcagcctgctgaaacaggccggcgacgtggaagagaaccctggccctatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagcatcacatttaaaagcatctcaggtaactatattttgaatttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttctgtacacgcgatccatcacactggcggccgctcgactggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 4121 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtaggatctttttccctctgccaaaaattatggggacatcatgaagccccttgagcatctgacttctggctaataaaggaaatttattttcattgcaatagtgtgttggaattttttgtgtctctcactcgatgaagtgggtaacctttatttcccttctttttctctttagctcggcttattccaggggtgtgtttcgtcgagatgcacgtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccagacaagagtgaggttgctcatcggtttaaagatttgggagaagaaaatttcaaagccttgtaagttaaaatattgatgaatcaaatttaatgtttctaatagtgttgtttattattctaaagtgcttatatttccttgtcatcagggttcagattctaaaacagtgctgcctcgtagagttttctgcgttgaggaagatattctgtatctgggctatccaataaggtagtcactggtcacatggctattgagtacttcaaatatgacaagtgcaactgagaaacaaaaacaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 8516 bp, Artificial Sequence
  synthetic
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcaagtttgtacaaaaaagcaggctgccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 39 - DNA, 8985 bp, Artificial Sequence
  synthetic
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 40 - DNA, 5864 bp, Artificial Sequence
  synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattcgagctcggtacctcgcgtgcatctagataatccaccatggcagctgcccttagagtcgccgcagtcggagctagactgagcgtgctcgcgtcaggtctgcgagccgctgtgaggagcctgtgcagccaggccacttccgtgaacgaacggattgagaacaagaggcggactgccctgctgggaggaggacagcggcgcatcgatgcacagcacaaacgcggcaagctgactgcccgcgaacggatctcccttctcctggatcccggctccttcgtggaatccgatatgtttgtggaacaccgctgcgccgactttggaatggcagccgacaagaacaagttccctggagactccgtggtgaccggtcggggacgcattaatggccgcctggtgtacgtgttcagccaggacttcactgtgttcggcggaagcctgagcggagcccacgctcagaagatctgcaagatcatggaccaggccatcacggtcggagctccggtcatcggcctcaacgactccggtggagcacggatccaagagggagtggaatcactggccggctatgcggacattttcctgagaaacgtgactgcctccggggtgatccctcagatctccctgatcatggggccctgtgctggaggagccgtgtactcaccggccctgaccgatttcaccttcatggtcaaggatacctcgtacctcttcattactggcccagacgtggtcaagtccgtgaccaacgaggacgtgacccaggaggagttgggaggcgccaagacacacaccaccatgagcggagtcgcccatcgggcgttcgagaacgatgtcgacgccctgtgcaatctgcgggatttctttaactacctcccgctgtccagccaagaccctgcacctgtgcgggaatgccacgacccaagcgacaggctggtgccggaactcgacaccattgtgcctctggaatcgaccaaggcgtacaacatggtcgatatcatccactctgtggtggatgaacgcgaattcttcgagatcatgccgaactacgccaagaatattatcgtgggatttgctcggatgaacggacgcactgtgggcattgtcggcaaccagcccaaggtcgcctccggatgcctggacattaactcctccgtgaagggcgcgaggtttgtgcgcttctgcgacgcctttaacattcccctgattaccttcgtcgatgtccccggattcctgcctggcaccgctcaggaatacgggggcatcatccgccatggcgccaagctgctctatgccttcgccgaagccaccgtgccaaaagtgactgtgatcacccggaaggcttacggcggtgcctatgacgtgatgtcgtcgaagcacttgtgtggcgacactaactatgcctggcccactgccgagatcgctgtgatgggagctaaaggggccgtggagatcatcttcaaggggcatgagaacgtggaagccgcccaagcggaatacattgagaagttcgcgaaccccttccctgctgccgtgagaggcttcgtggacgacatcatccagccgagctcgaccagagcgagaatctgctgcgacctcgacgtcctggcgtccaagaaggtccaacggccttggaggaagcatgcaaacatcccgctgtagagacgcgtggtacctctagagtcgacccgggcggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagacggtcagactaaactggctgacggaatttatgcctcttccgaccatcaagcattttatccgtactcctgatgatgcatggttactcaccactgcgatccctggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcac